(19) United States
(12) Patent Application Publication
(10) Pub. No.: US 2018/0140545 A1
(43) Pub. Date: May 24, 2018

(54) ANTIMICROBIAL PEPTIDE STIMULATING SANITIZING COMPOSITION

(71) Applicant: GOJO Industries, Inc., Akron, OH (US)

(72) Inventors: Kegui Tian, Hudson, OH (US); Jessica Rae Tittl, Akron, OH (US)

(21) Appl. No.: 15/819,230

(22) Filed: Nov. 21, 2017

Related U.S. Application Data
(60) Provisional application No. 62/425,743, filed on Nov. 23, 2016.

(51) Int. Cl. A61K 9/00; A61K 31/047; A61K 38/08; A61K 36/87

(52) U.S. Cl. CPC A61K 9/0043; A61K 36/87; A61K 38/08; A61K 31/047

(57) ABSTRACT

A method of increasing antimicrobial peptide concentration on the skin is provided. The method includes cleaning skin with at least one of a cleanser and a sanitizer and applying a sanitizing composition to the skin. The sanitizing composition comprises one or more polypeptides and extracts that increase the concentration of antimicrobial peptides.

### RELATED APPLICATIONS

This application claims priority to, and the benefits of, U.S. Provisional Pat. App. No. 62/425,743, titled ANTIMICROBIAL PEPTIDE STIMULATING SANITIZING COMPOSITION, filed on Nov. 23, 2016, which is incorporated herein in its entirety.

### BACKGROUND

Skin disinfecting or sanitizing compositions have become increasingly popular in the health care industry as well as with the general public for providing antimicrobial effectiveness to the skin without irritation. Generally, these skin disinfecting or sanitizing compositions, which should be distinguished from skin cleansing compositions such as soaps, shampoos, and detergents which typically include surfactants, abrasives, or other active ingredients used to physically as well as microscopically cleanse the skin, include alcohol which kills a wide array of microorganisms which may be present on the skin, particularly the hands.

Recent microbiome studies have analyzed the chemical make-up of the skin and the potential for sanitizing compositions to improve both skin defense against germs and skin's innate immunity. This includes germ control through both internal and external methods. External methods include hygiene products that directly kill or slow germ growth. Internal methods include improving an organism's immune system to fight germs itself.

Antimicrobial peptides (“AMPs”), also known as host defense peptides, comprise a wide range of natural and synthetic peptides that are made of oligopeptides containing a varying number of amino acids. AMPs are essential components of host defense against infections present in all domains of life. AMPs are produced by all complex organisms and have diverse and intricate antimicrobial activities. As a whole, these peptides demonstrate a broad range of antiviral and antibacterial activities through an array of modes of action. AMPs have been found to kill Gram-negative and Gram-positive bacteria, certain viruses, parasites and fungi. Some research suggests that they can also enhance the internal immunity of complex organisms against a broad range of bacteria and viruses. In addition to the innate immune system present in all animals, vertebrates evolved an adaptive immune system based on specific recognition of antigens. Increasing evidence suggests that AMPs released in response to an invasion of microbial can activate adaptive immunity by attracting antigen-presenting dendritic cells to the invasion site.

### SUMMARY

According to some exemplary embodiments, a sanitizing composition for increasing the production and/or activity of antimicrobial peptides is provided. The sanitizing composition includes about 0.005 wt. % to about 15.0 wt. % of an active ingredient that is one or more of an extract and a polypeptide. The sanitizing composition also includes one or more ingredients that deliver a sanitizing effect. The application of the sanitizing composition increases the production and/or activity of antimicrobial peptides on the surface of the skin by an amount that is statistically significant compared to an otherwise identical sanitizing composition without the active ingredient.

In some exemplary embodiments, the one or more ingredients that deliver a sanitizing effect is an anti-microbial agent that can be one or more of an alcohol and a quaternary ammonium compound. The alcohol can be one or more C₁₋₈ alcohols, such as methanol, ethanol, propanol, pentanol, hexanol, and isomers and mixtures thereof. In some exemplary embodiments, the alcohol is included in an amount above about 70.0 wt. %, based on the weight of the sanitizing composition.

In some exemplary embodiments, the active ingredient is an extract that is one or more of a plant extract, a seed extract and a fruit extract. In other embodiments, the seed extract is at least one of linseed extract, flaxseed extract, hemp seed extract, grape seed extract, and grapefruit seed extract.

In some exemplary embodiments, the active ingredient is a hydrolysate of proteins, which can be proteins extracted from linseed seeds. The hydrolysate of linseed proteins can contain from about 0.1 to about 5.0 g/l of peptide compounds and from about 0.1 to 2.0 g/l of sugar. The peptide compounds can have a molecular weight below about 5 kDa.

In some exemplary embodiments, the active ingredient is a polypeptide that is one or more of an oligopeptide and a hexapeptide.

In some exemplary embodiments, the sanitizing composition comprises from about 0.05 to about 5.0 wt. % or from about 0.1 to about 1.0 wt. % of the active ingredient, based on the weight of the sanitizing composition.

In some exemplary embodiments, the sanitizing composition further comprises one or more skin conditioning agents.

In some exemplary embodiments, the sanitizing composition contains up to about 20.0 wt. % of a humectant, based on the weight of the sanitizing composition, as the skin conditioning agent, comprising propylene glycol, hexylene glycol, 1,4-dihydroxyhexane, 1,2,6-hexanetriol, sorbitol, butylene glycol, caprylyl glycol, propanediols, such as methyl propane diol, dipropylene glycol, triethylene glycol, glycerin (glycerol), polyethylene glycols, ethoxydiglycol, polyethylene sorbitol, glyceryl caprylate/caprate, and combinations thereof.

In some exemplary embodiments, the sanitizing composition also contains up to 10.0 wt. % of a moisturizing ester, based on the weight of the sanitizing composition, comprising cetyl myristate, cetyl myristoleate, and other cetyl esters, diisopropyl sebacate, isopropyl myristate, and combinations thereof.

In some exemplary embodiments, the sanitizing composition increases the production and/or activity of at least one anti-microbial peptide by a statistically significant amount. In some exemplary embodiments, the sanitizing composition increases the production and/or activity of defenins by at least about 7%, or at least about 18%, or at least about 20%, or at least about 1 pg/mL or at least about 4 pg/mL. In some exemplary embodiments, the sanitizing composition increases the production and/or activity of chemokines by at least about 30%. In some exemplary embodiments, the sanitizing composition increases the production and/or activity of cathelicidin-related antimicrobial peptides by at least about 32%. All percentages are relative to an otherwise identical sanitizing composition without the active ingredient.

In some exemplary embodiments, the sanitizing composition further comprises a carrier, which can be water.

In another exemplary embodiment, a skin treatment method for increasing the production and/or activity of antimicrobial peptides is provided. The method includes applying a sanitizing composition to a skin surface, wherein the sanitizing composition includes about 0.005 wt. % to about 15.0 wt. % of an active ingredient. The active ingredient may be one or more of an extract and a polypeptide. The sanitizing composition also includes one or more ingredients that deliver a sanitizing effect. The application of the sanitizing composition increases the production and/or activity of AMPs on the surface of the skin by an amount that is statistically significant compared to an otherwise identical composition without the active ingredient.

In another exemplary embodiment, a skin treatment composition is provided. The skin treatment composition comprises about 0.005 wt. % to about 15.0 wt. % of an active ingredient comprising one or more of an extract and a polypeptide, about 40.0 wt. % to about 95.0 wt. % of one or more ingredients that deliver a sanitizing effect, about 0.01 wt. % to about 10.0 wt. % of one or more skin conditioners, and about 0.01 wt. % to about 5.0 wt. % of a moisturizing ester.

In another exemplary embodiment, a sanitizing composition for increasing the innate immunity of the skin is provided. The sanitizing composition comprises about 0.005 wt. % to 15.0 wt. % of an active ingredient comprising one or more of an extract and a polypeptide, about 40.0 wt. % to about 95.0 wt. % of one or more ingredients that deliver a sanitizing effect, about 0.01 wt. % to about 5.0 wt. % of one or more skin conditioners, and about 0.01 wt. % to about 5.0 wt. % of a viscosity modifier.

In another exemplary embodiment, a sanitizing composition for increasing the innate immunity of the skin is provided. The sanitizing composition comprises about 0.005 wt. % to 15.0 wt. % of an active ingredient comprising one or more of an extract and a polypeptide, about 40.0 wt. % to about 95.0 wt. % of one or more ingredients that deliver a sanitizing effect, about 0.01 wt. % to about 5.0 wt. % of a foaming agent, and about 0.01 wt. % to about 10.0 wt. % of one or more skin conditioners.

According to some exemplary embodiments, a nasal spray sanitizing composition is provided. The nasal spray comprises one or more C₁₋₈ alcohols and about 0.005 wt. % to about 15.0 wt. % of an active ingredient that is one or more of a probiotic, a probiotic derivative, and a prebiotic. Application of the nasal spray reduces pathogen binding in the nose by an amount that is statistically significant compared to an otherwise identical nasal spray without the active ingredient.

In some exemplary embodiments, the nasal spray further comprises one or more fragrances.

### BRIEF DESCRIPTION OF THE FIGURES

FIG. 1 graphically illustrates HBD-1 concentrations after treatment with various concentrations of Decorinyl and Pamitoyl Pentapeptide-3.

FIG. 2 graphically illustrates HBD-2 concentrations after treatment with various concentrations of Decorinyl and Pamitoyl Pentapeptide-3.

FIG. 3 graphically illustrates HBD-3 concentrations after treatment with various concentrations of Decorinyl and Pamitoyl Pentapeptide-3.

FIG. 4 graphically illustrates HBD-1 concentrations after treatment with 0.1% and 1.0% Lipigenine™.

FIG. 5 graphically illustrates HBD-2 concentrations after treatment with 0.1% and 1.0% Lipigenine™.

FIG. 6 graphically illustrates HBD-3 concentrations after treatment with 0.1% and 1.0% Lipigenine™.

FIG. 7 graphically illustrates LL-37 concentrations after treatment with 0.1% and 1.0% Lipigenine™.

FIG. 8 graphically illustrates IL-8 concentrations after treatment with 0.1% and 1.0% Lipigenine™.

FIG. 9 graphically illustrates HBD-1 concentrations after treatment with various ingredients.

FIG. 10 graphically illustrates HBD-2 concentrations after treatment with various ingredients.

FIG. 11 graphically illustrates HBD-3 concentrations after treatment with various ingredients.

FIG. 12(a) graphically illustrates the HBD-1 concentration after the addition of Lipigenine™ to the PURELL Advanced gel sanitizer.

FIG. 12(b) graphically illustrates the HBD-1 concentration after the addition of Lipigenine™ to the PURELL Advanced gel sanitizer.

FIG. 13(a) graphically illustrates the HBD-2 concentration after the addition of Lipigenine™ to the PURELL Advanced gel sanitizer.

FIG. 13(b) graphically illustrates the HBD-2 concentration after the addition of Lipigenine™ to the PURELL Advanced gel sanitizer.

FIG. 14 graphically illustrates the HBD-3 concentration after the addition of Lipigenine™ to the PURELL Advanced gel sanitizer.

### DETAILED DESCRIPTION

Unless otherwise defined, all technical and scientific terms used herein have the same meaning as commonly understood by one of ordinary skill in the art to which this application pertains. Although other methods and materials similar or equivalent to those described herein may be used in the practice or testing of the exemplary embodiments, exemplary suitable methods and materials are described below. In case of conflict, the present specification including definitions will control. In addition, the materials, methods, and examples are illustrative only and not intended to be limiting of the general inventive concepts.

The terminology as set forth herein is for description of the exemplary embodiments only and should not be construed as limiting the application as a whole. Unless otherwise specified, “a,” “an,” “the,” and “at least one” are used interchangeably. Furthermore, as used in the description of the application and the appended claims, the singular forms “a,” “an,” and “the” are inclusive of their plural forms, unless contradicted by the context surrounding such.

The phrase “statistically significant” means p<0.05 for a test composition vs. a control that does not contain the active ingredient. The analysis is completed using 1) a T-test (a statistical examination of two population means) when only comparing one test article vs. one control); or 2) an analysis of variance (ANOVA) test when comparing two or more test articles vs. controls.

The terms “polypeptide” and “polypeptides” as used herein refer to a chain of amino acids with two or more peptide bonds. In this way, these terms are meant to encompass both oligopeptides (which are generally considered to be peptide chains with between two and ten amino acids) as well as polypeptides (which are generally considered to be peptide chains with more than 10 amino acids).

The general inventive concepts relate to a sanitizing composition that contains an AMP-stimulating active ingredient, including an extract and/or one or more polypeptides. In some exemplary embodiments, the active ingredient is an extract. The extract can be a modified extract, an unmodified extract, or an extract derivative. In one exemplary embodiment, the active ingredient is a natural extract, and can be derived from a plant extract, a fruit extract, and/or a seed extract. Non-limiting examples of natural extracts may include seed extracts, fruit extracts, linseed extract, flaxseed extract, hemp seed extract, grape seed extract, grapefruit seed extract, watermelon fruit extract, apple fruit extract, lentil fruit extract, hibiscus flower extract, pear fruit extract, root extract, leaf extract, Schinus terebinthifolius Seed Extract, Ascophyllum nodosum extract, soybean extract, Crothmum martimum extract, Lavandula stoechas extract, stem extracts, Sapindus Mukurossi fruit extract, sandalwood extract, bark extract, barley extract, Polygonum fagopyrum seed extract, avocado extract, cranberry fruit extract, blueberry fruit extract, Silena uniforla extract, Rosa multiflora extract, Evodia rutaecarpa fruit extract, algae extract, licorice leaf extract, jobi seed extract, seed oils, rosemary extract, green tea extract, plankton extract, himanthalia elongata extract, unidaria pinnatifida extract, Chlorella vulgaris extract, mugwort extract, and the like.

In some exemplary embodiments, the extract can be produced from the hydrolysis of natural proteins, which is referred to as a hydrolysate of proteins. Thus, the natural extracts may themselves comprise one or more peptides and/or polypeptides, or the active ingredient may comprise peptides and/or polypeptide(s) independently. The hydrolysate can be obtained through hydrolysis of any type of protein, including proteins from any source. In some exemplary embodiments, the extract is a hydrolysate of linseed proteins, which are the proteins extracted from linseed seeds. Preferably, the linseed extract contains from about 0.1 to about 5.0 g/l of peptide compounds by weight of the dry extract and from about 0.1 to about 2.0 g/l of sugar by weight of the dry extract. These peptide compounds preferably have a molecular weight below about 5.0 kDa or below about 2.5 kDa.

The proteins can be any type of protein and can come from any type or part of a plant. In some exemplary embodiments, the plant can be of the Malpighiales order, of the Liaceae family, and/or of the Linum genus (linseed). Any method of extraction and purification can be employed to procure and prepare the protein extract.

In some exemplary embodiments, the natural extract is selected from one or more of the following compositions: (1) glycerin, plantago lanceolata leaf extract and xanthan gum (sold under the trade name Senestem™ by Sederma); (2) Benoitine (plankton extract in water); (3) water, glycerin, and hydrolyzed pearl (sold under the trade name Crodarom® by Croda Inc.) (4) Red Bush (rooibos) plant extract, (5) Phyko-Al-PF (water and hydrolyzed algin), and water, glycerin, and linseed (linum usitatissimum) seed extract (sold under the trade name Lipigenine™ by Ashland Chemical Company).

In some exemplary embodiments, the active ingredient comprises one or more peptides. Peptides are biologically-occurring short chains of amino acid monomers joined together by amide (peptide) bonds, which are formed through condensation reactions.

In other exemplary embodiments, the active ingredient comprises one or more oligopeptides. Oligopeptides are generally defined as peptide chains with 10 or fewer amino acids. In this way, the oligopeptide may be include, but is not limited to, an oligopeptide, such as a dipeptide, a tripeptide, a tetrapeptide, a pentapeptide, a hexapeptide, a heptapeptide, an octapeptide, a nonapeptide, and a decapeptide.

In other exemplary embodiments, the active ingredient comprises one or more polypeptides. A polypeptide is a long, continuous, unbrached peptide chain. Polypeptides are generally defined as peptide chains with more than 10 amino acids. The polypeptides of the exemplary embodiments described herein are not particularly limited and can be made of any number of peptide bonds.

In other exemplary embodiments, the active ingredient comprises a protein, which includes at least one long polypeptide that is arranged in a biologically functional way. The proteins of the exemplary embodiments described herein are not particularly limited and can include any number of polypeptides arranged in any biologically active manner. The peptides, oligopeptides, polypeptides, and proteins comprising the subject sanitizing composition can be natural or synthetic peptides or polypeptides. They can further be modified or unmodified.

Exemplary polypeptides include Juvefoxo™; tetrapeptides, such as Uplevity™, Relistase®, and Decorinyl®; pentapeptides, such as palmitoyl pentapeptide-4, palmitoyl pentapeptide-3, and acetyl pentapeptide-1; hexapeptides, such as Adifyline® and acetyl hexapeptides; and mixtures of polypeptides and natural extracts, such as Triple A Complex, Trylagen® PCB. Exemplary acetyl hexapeptides include acetyl hexapeptide-1, acetyl hexapeptide-3, acetyl hexapeptide-7, acetyl hexapeptide-8, acetyl hexapeptide-19, acetyl hexapeptide-20, acetyl hexapeptide-22, acetyl hexapeptide-24, acetyl hexapeptide-30, acetyl hexapeptide-31, acetyl hexapeptide-37, acetyl hexapeptide-38, acetyl hexapeptide-39, acetyl hexapeptide-46, and acetyl hexapeptide-49. In some exemplary embodiments, the polypeptides include two or more acetyl hexapeptides.

In some exemplary embodiments, the sanitizing composition according to the exemplary embodiments described herein includes an effective amount of active ingredient to increase the production and/or activity of at least one antimicrobial peptide on, for example, the skin. The sanitizing composition can increase the production and/or activity of a wide variety of antimicrobial peptides, such as, for example defensins and cathelicidin-related AMPs and decrease pro-inflammatory factors. Such increased production and/or activity helps the skin's ability to defend against germs and helps improve the skin's innate immunity.

In one exemplary embodiment, the sanitizing composition increases the production and/or activity of defensins. Defensins are cationic proteins that function as host defense peptides that have been found in vertebrates, invertebrates, and some plants. Defenins include at least α-defensins, β-defensins, and θ-defensins. In some exemplary embodiments, the sanitizing composition increases the production and/or activity of β-defensins, such as HBD-1, HBD-2, and HBD-3.

In some exemplary embodiments, the sanitizing composition increases the production and/or activity of cathelicidin-related antimicrobial peptides. Cathelicidins play a vital role in mammalian innate immunity against invasive bacterial infections. In some exemplary embodiments, the sanitizing composition increases the production and/or activity of the cathelcidin-related AMP, LL-37.

In other exemplary embodiments, the sanitizing composition decreases the production and/or activity of pro-inflammatory factors. One such pro-inflammatory factor is cytokines, which are a group of small proteins that are involved in cell signaling. There are numerous groups of cytokines including chemokiens, interferons, interleukins, lymphokines, and tumor necrosis factors. Interleukins are a group of cytokines and include 17 different families, interleukins 1-17. In some exemplary embodiments, the sanitizing composition increases the production and/or activity of the pro-inflammatory factor, cytokines. In some exemplary embodiments, the sanitizing composition increases the production and/or activity of the cytokine, interleukins, such as interleukin-8 (IL-8).

Traditionally, it has been found that compositions used to stimulate the production and/or activity of AMPs also cause skin inflammation and/or skin irritation. However, it has been discovered that a sanitizing composition comprising the subject active ingredient is capable of increasing the production and/or activity of at least one AMP on the skin without causing irritation/inflammation of the skin.

The effective amount of active ingredient in the sanitizing composition may include up to about 15.0 percent by weight (wt. %) of the active ingredient, based on the total weight of the sanitizing composition. In some exemplary embodiments, the effective amount of active ingredient comprises from about 0.005 to about 15.0 wt. %, or from about 0.02 to about 5.0 wt. %, or from about 0.5 to about 2.0 wt. %, based on the weight of the sanitizing composition. In other exemplary embodiments, the effective amount of active ingredient comprises about 0.1 to about 1.0 wt. %, based on the weight of sanitizing composition.

In some exemplary embodiments, the sanitizing composition is a sanitizing composition used for application to surfaces, such as the human skin. In some other exemplary embodiments, the sanitizing composition is used for application in various human orifices, such as the nose. While surfaces on the human body, such as the skin or nose are discussed herein, it is to be appreciated that the compositions and methods disclosed herein can be used on non-mammalian and inanimate objects and surfaces.

The sanitizing composition may be in the form of a gel, a foam, a salve, a wipe, a cream, etc. A wide variety of vehicles may be used to deliver the sanitizing composition, such as, for example pads, bandages, patches, sticks, aerosol dispersers, pump sprays, trigger sprays, canisters, foam pumps, wipes, and the like. The sanitizing composition may be applied to the skin before, during, or after skin cleaning. In some exemplary embodiments, the sanitizing composition is applied after skin cleaning.

In some exemplary embodiments, the sanitizing composition comprises one or more ingredients that have a sanitizing effect. The term “ingredient with a sanitizing effect” is meant to include any compound, ingredient, molecule, or combination or blend thereof that achieves at least a 2-log reduction in the number of viable microorganisms in vitro after 60 seconds according to the ASTM E2783 11(2016) time kill test method. The ingredient with a sanitizing effect can be an anti-microbial agent. Anti-microbial agents are compounds that kill and/or protect against the growth of microorganisms.

In some exemplary embodiments, the ingredient with a sanitizing effect is one or more of alcohol, povidone-iodine, triclosan, triclocarban, chlorohexidine, chlorine, hexachlorophene, iodine, chloroxyenol, chlorine dioxide, oxidizing agents, polyhexamethylene biguanide (PHMB), hydrogen peroxide, phenoxyethanol, iodine, antimicrobial peptides, hypochlorites, lysozymes, alkyl gallates, quinones, catechins, urea, biguanide, oxygen, and quaternary ammonium compounds such as benzalkonium chloride or benzethonium chloride. In some exemplary embodiments, the ingredient with a sanitizing effect is an aldehyde donor, such as a formaldehyde donor. In some exemplary embodiments the ingredient with a sanitzing effect is a surfactant. In some exemplary embodiments, the surfactant is an anionic surfactant, a cationic surfactant, or a nonionic surfactant.

In some exemplary embodiments, the ingredient with a sanitizing effect is an acid. In some exemplary embodiments, the acid is an organic acid. In some exemplary embodiments, the acid is one or more of citric acid, lactic acid, hypochlorous acid, caffeic acid, and the like. In some exemplary embodiments, the acid is a peracid, such as peracitic acid, perlactic acid, peroctanoic acid, perbenzoic acid, peracetic acid, perpropionic acid, performic acid, and the like. The peracid can be an inorganic or organic peracid. In some exemplary embodiments, the ingredient with a sanitizing effect is an essential oil and/or derivative thereof. Non-limiting examples of such compounds include carvacrol, thymol, linalool, farnesol, and the like. In some exemplary embodiments, the ingredient with a sanitizing effect is a carboxylic acid, such as a mono-, di-, tri-, or tetra-carboxylic acid or a polymeric carboxylic acid.

In some exemplary embodiments, the ingredient with a sanitizing effect is a compound that contains one or more of silver and copper or alloys thereof (such as brasses, bronzes, cupronickel, and the like). In some exemplary embodiments, the silver or copper has been ionized. In some exemplary embodiments, the ingredient with a sanitizing effect is elemental copper or elemental silver. In some exemplary embodiments, the ingredient with a sanitizing effect is a silver salt or copper salt. In some other exemplary embodiments, the ingredient with a sanitizing effect is a titanium dioxide based solution.

In some exemplary embodiments, the ingredient with a sanitizing effect is an organic peroxide, a phenolic compound, a free radical, or a glycol. In some exemplary embodiments, the ingredient with a sanitizing effect is an ion, such as a hydroxyl ion or a metal ion. In some exemplary embodiments, the ingredient with a sanitizing effect is any compound that has been identified or approved by the United States Food and Drug Administration as being suitable as an active ingredient/anti-microbial agent in a sanitizing composition.

In some exemplary embodiments, the ingredient that has a sanitizing effect in the sanitizing composition is an alcohol or combination of alcohols. By alcohol, it is meant any organic compound which has a hydroxyl functional group bonded to a saturated carbon atom. Alcohol has antimicrobial properties and has the ability to kill many forms of bacteria, fungi, and viruses. In some embodiments, the alcohol is a C₁₋₈ alcohol, i.e. an alcohol containing 1 to 8 carbon atoms. Such alcohols may be referred to as lower alkanols. Examples of lower alkanols include, but are not limited to, methanol, ethanol, propanol, butanol, pentanol, hexanol, and isomers and mixtures thereof. The alcohol may be either pure alcohol or denatured alcohol. In one or more exemplary embodiments, the alcohol comprises ethanol, propanol, or butanol, or isomers or mixtures thereof. In one or more exemplary embodiments, the alcohol comprises isopropanol. In other exemplary embodiments, the alcohol comprises ethanol. In one or more exemplary embodiments, the sanitizing composition comprises a mixture of alcohols. In one or more exemplary embodiments, the sanitizing composition comprises a mixture of ethanol and isopropanol. In one or more exemplary embodiments, the sanitizing composition comprises a mixture of isopropanol and n-propanol. In one exemplary embodiment, the sanitizing composition comprises ethanol.

While C₁₋₈ alcohols are discussed herein as able to deliver the necessary sanitizing effect, it is envisioned that longer alcohols (i.e., alcohols with more than 8 carbon atoms), or alcohols with various other functional groups would be similarly suitable. For example, in addition to the hydroxyl functional group, the alcohol may further contain esters, carboxylic acids, ethers, amides, amines, alkyl halides, phenyls, as well as other carbonyl-containing functional groups. The alcohol can also be an aliphatic alcohol or an aromatic alcohol.

Generally, the sanitizing composition may comprise at least about 1.0 wt. % C₁₋₈ alcohol, based on the total weight of the composition. In one embodiment, the sanitizing composition comprises at least about 2.0 wt. % C₁₋₈ alcohol, in another embodiment, the sanitizing composition comprises at least about 10.0 wt. % C₁₋₈ alcohol, in another embodiment, the sanitizing composition comprises at least about 20.0 wt. % C₁₋₈ alcohol, in another embodiment, the sanitizing composition comprises at least about 40.0 wt. % C₁₋₈ alcohol, in another embodiment, the sanitizing composition comprises at least about 50.0 wt. % C₁₋₈ alcohol, in another embodiment, the sanitizing composition comprises at least about 60.0 wt. % C₁₋₈ alcohol, in another embodiment, the sanitizing composition comprises at least about 65.0 wt. % C₁₋₈ alcohol, in yet another embodiment, the sanitizing composition comprises at least about 70.0 wt. % C₁₋₈ alcohol, and in still yet another embodiment, the sanitizing composition comprises at least about 80.0 wt. % C₁₋₈ alcohol, based on the weight of the sanitizing composition. In other embodiments, the sanitizing composition comprises from about 60.0 to about 95.0 wt. % C₁₋₈ alcohol, based on the weight of the sanitizing composition. In other exemplary embodiments, the sanitizing composition comprises from about 73.0 to about 78.0 wt. % C₁₋₈ alcohol, based on the weight of the sanitizing composition. More or less alcohol may be required in certain instances, depending particularly on other ingredients and/or the amounts thereof employed in the sanitizing composition.

In other exemplary embodiments, where the ingredient with a sanitizing effect is not alcohol, the amount of the ingredient is not particularly limited. In some exemplary embodiment, the ingredient with a sanitizing effect can be added in an amount up to about 95.0 wt. %, or about 85.0 wt. %, or about 75.0 wt. %, or about 65.0 wt. %, based on the total weight of the sanitizing composition. In other exemplary embodiments, the ingredient with a sanitizing effect can be added in an amount as low as about 0.001 wt. %, or about 0.01 wt. %, or about 0.1 wt. %. In some exemplary embodiments, the ingredient with a sanitizing effect can be added in an amount from about 0.01 to about 15.0 wt. %, based on the total weight of the sanitizing composition. In some exemplary embodiments, the ingredient with a sanitizing effect is added in an amount from about 0.5 to about 7.5 wt. %, or about 0.75 to about 5.0 wt. %, or from about 1.0 to about 4.0 wt. %, based on the total weight of the sanitizing composition.

The active ingredient's ability to stimulate the production of AMPs in an alcohol-based solution was particularly surprising. Typically, when alcohol is added in the presence of a protein (polypeptide), the protein is denatured and loses its secondary and tertiary structures. Specifically, the normal alpha-helix and beta sheets in the protein are uncoiled and the protein is sent into a random shape. In this way the protein is no longer able to function as intended. The alcohol causes this undesirable reaction because it disrupts the hydrogen bonding in the protein. Hydrogen bonds are electostatic attractions between polar groups occurring when a hydrogen atom is bonded to a highly electronegative atom (typically oxygen or nitrogen). It the case of proteins, hydrogen bonding occurs between amide groups (which include a nitrogen atom) in the secondary protein structure as well as the tertiary protein structure in certain amino acids. Because of the OH group in the alcohol (which can also hydrogen bond with the protein), the internal hydrogen bonding within the protein is disrupted. Due to the particular structures of the active ingredients described herein, the molecules avoid the disruptive reactions detailed above.

In some exemplary embodiments, the sanitizing composition comprises a carrier. The carrier can be any suitable compound able to effectively deliver and/or transport the sanitizing composition. In some exemplary embodiments, the carrier is water or a base cleaner. In other exemplary embodiments, the sanitizing composition does not include any carrier and is delivered as a concentrate.

In some exemplary embodiments, the sanitizing composition includes water as the carrier in an amount quantum sufficit (q.s.). In some exemplary embodiments, the sanitizing composition comprises at least about 1.0 wt. % water, in another embodiment the sanitizing composition comprises at least about 10.0 wt. % water, in another embodiment, the sanitizing composition comprises at least about 20.0 wt. % water, in another embodiment, the sanitizing composition comprises at least about 30.0 wt. % water, in another embodiment, the sanitizing composition comprises at least about 40.0 wt. % water, in another embodiment, the sanitizing composition comprises at least about 50.0 wt. % water, and in yet another embodiment, the sanitizing composition comprises at least about 60.0 wt. % water, and in still yet another embodiment, the sanitizing composition comprises at least about 70.0 wt. % water, based on the weight of the sanitizing composition. In other embodiments, the sanitizing composition comprises from about 20.0 wt. % to about 30.0 wt. % water. In a preferred embodiment, the sanitizing composition comprises from about 20.0 to about 24.0 wt. % water, based on the weight of the sanitizing composition. More or less water may be required in certain instances, depending particularly on other ingredients and/or the amounts thereof employed in the sanitizing composition.

In one or more embodiments, the sanitizing composition includes one or more skin-conditioners. Various classes or types of skin-conditioners have been used such as humectants, emollients, and other miscellaneous compounds which exhibit occlusive properties upon application to the skin. Non-limiting examples of suitable skin conditioners and emollients include aloe, vitamin E, vitamin E acetate (tocopheryl acetate), Vitamin B₃ (niacinamide), C₆₋₁₀ alkane diols, sodium salt of pyroglutamic acid (sodium PCA), PEG-7 glyceryl cocoate, coco-glucoside and/or glyceryl oleate (Lamisoft® PO), and polyquaternium, such as polyquaternium 10 and 39.

If an emollient or one of the miscellaneous skin-conditioners, such compound can be included in the sanitizing composition in an amount from about 0.0001 to about 10.0 wt. %, in other embodiments, from about 0.0005 to about 5.0 wt. %, based on the weight of the sanitizing composition. In one exemplary embodiment, the miscellaneous skin conditioner is present in an amount from about 0.1 to about 2.0 wt. %, based on the total weight of the composition and in yet another exemplary embodiment, from about 0.5 to about 1.0 wt. %, based on the weight of the sanitizing composition.

In some exemplary embodiments, the sanitizing composition includes one or more humectants as the skin conditioner. Non-limiting examples of humectants include propylene glycol, hexylene glycol, 1,4-dihydroxyhexane, 1,2,6-hexanetriol, sorbitol, butylene glycol, caprylyl glycol, propanediols, such as methyl propane diol, dipropylene glycol, triethylene glycol, glycerin (glycerol), polyethylene glycols, ethoxydiglycol, polyethylene sorbitol, and combinations thereof. Other humectants include glycolic acid, glycolate salts, lactate salts, urea, hydroxyethyl urea, alpha-hydroxy acids, such as lactic acid, sodium pyrrolidone carboxylic acid, hyaluronic acid, chitin, glyceryl caprylate/caprate (GCC), and the like. In one exemplary embodiment, the humecant is a mixture of caprylyl glycol and glycerin.

Examples of polyethylene glycol humectants include PEG-4, PEG-6, PEG-7, PEG-8, PEG-9, PEG-10, PEG-12, PEG-14, PEG-16, PEG-18, PEG-20, PEG-32, PEG-33, PEG-40, PEG-45, PEG-55, PEG-60, PEG-75, PEG-80, PEG-90, PEG-100, PEG-135, PEG-150, PEG-180, PEG-200, PEG-220, PEG-240, and PEG-800.

The humectant may be included in the sanitizing composition in an amount up to about 20.0 wt. %, or up to about 15.0 wt. %, or up to about 12.0 wt. %, or up to about 10.0 wt. %, or up to about 8.0 wt. % or up to about 8.0 wt. %, or up to about 3.0 wt. %, based on the weight of the sanitizing composition. In some exemplary embodiments, the humectant is included in an amount from about 0.001 wt. %, or from about 0.01 wt. %, or from about 0.05 wt. %, or from about 0.1 wt. %, or from about 0.5 wt. %, or from about 0.7 wt. %, or from about 1.0 wt. %, or from about 1.5 wt. %, or from about 2.0 wt. %, based on the weight of the sanitizing composition. In one exemplary embodiment, the humectant is included in an amount from about 0.4 to about 3.0 wt. %, based on the weight of the sanitizing composition.

The sanitizing composition may further comprise a plug-preventing additive. In some exemplary embodiments, the plug-preventing additive can also, as discussed above, act as the humectant. In one or more embodiments the plug-preventing comprises one or more diols, that is compounds with two hydroxyl groups. Plug-preventing additives that contain more or less hydroxyl groups (i.e., one hydroxyl group or three or more hydroxyl groups) are also within the purview of the exemplary embodiments described herein. In one or more exemplary embodiments the diol is a C₆₋₁₀ alkane diol and in some exemplary embodiments, a straight chain C₆₋₁₀ alkane diol, that is, a straight chain diol with a chain of 6 to 10 carbon atoms. Non-limiting examples of suitable diols include 1,2-hexanediol, 1,2-octanediol (often referred to as caprylyl glycol), 1,9-nonanediol, 1,2-decanediol, 1,10-decanediol, or mixtures and blends thereof. The diol can contain any other functional groups including, for example, esters, carboxylic acids, ethers, amides, amines, alkyl halides, phenyls, as well as other carbonyl-containing functional groups. In some exemplary embodiments, the plug-preventing agent contains at least one ester and/or at least one amide group. Non-limiting examples of such compounds include glyceryl caprylate/caprate and cocoamide diethanolamine.

If separate from the humectant, the plug-preventing additive may be included in the sanitizing composition in an amount up to about 20.0 wt. %, or up to about 15.0 wt. %, or up to about 12.0 wt. %, or up to about 10.0 wt. %, or up to about 8.0 wt. % or up to about 5.0 wt. %, or up to about 3.0 wt. %, based on the weight of the sanitizing composition. In some exemplary embodiments, the plug-preventing agent is included in an amount from about 0.001 wt. %, or from about 0.01 wt. %, or from about 0.05 wt. %, or from about 0.1 wt. %, or from about 0.5 wt. %, or from about 0.7 wt. %, or from about 1.0 wt. %, or from about 1.5 wt. %, or from about 2.0 wt. %, based on the weight of the sanitizing composition. In one exemplary embodiment, the plug-preventing additive is included in an amount from about 0.05 to about 4.0 wt. %, or from about 0.1 to about 1.0 wt. %, or from about 0.15 to about 0.7 wt. %, or from about 0.2 to about 0.7 wt. %, based on the weight of the sanitizing composition.

In certain embodiments, the diol plug-preventing additive is added to the sanitizing composition as a solution or emulsion. That is, the diol can be premixed with a carrier to from a diol solution or emulsion, with the proviso that the carrier does not deliriously effect the ability of the sanitizing composition to sanitize and increase the production or activity of antimicrobial peptides. Non-limiting examples of carriers include, water, alcohol, glycols such as propylene or ethylene glycol, ketones, linear and/or cyclic hydrocarbons, triglycerides, carbonates, silicones, alkenes, esters such as acetates, benzoates, fatty ester, glyceryl esters, ethers, amides, polyethylene glycol, and PEG/PPG copolymers, inorganic salts solutions such as saline, and mixtures and blends thereof.

The sanitizing composition may further comprise one or more conditioning or moisturizing esters. Examples of such conditioning or moisturizing esters include cetyl myristate, cetyl myristoleate, and other cetyl esters, diisopropyl sebacate, and isopropyl myristate. The ester may be present in an amount of up to about 10.0 wt. %, or up to about 8.0 wt. %, or up to about 5.0 wt. %, or up to about 3.0 wt. %, or up to about 2.0 wt. %, or up to about 1.0 wt. %, based on the weight of the sanitizing composition. In some exemplary embodiments, the moisturizing ester is present in an amount from about 0.001 wt. %, or from about 0.005 wt. %, or from about 0.01 wt. %, or from about 0.05 wt. %, or from about 0.1 wt. %, or from about 0.5 wt. %, or from about 1.0 wt. %, based on the weight of the sanitizing composition. In one exemplary embodiment, the moisturizing ester is present in an amount between 0.01 to 0.30 wt. %, based on the weight of the sanitizing composition. In another exemplary embodiment, the moisturizing ester is present in an amount between 0.05 wt. % and 0.25 wt. %, based on the weight of the sanitizing composition.

In one or more embodiments, the sanitizing composition includes one or more emulsifying agents. Examples of emulsifying agents include stearyl alcohol, sorbitan oleate trideceth-2, poloxamers, and PEG/PPG-20/6 dimethicone. In some exemplary embodiments, the emulsifying agent is present in an amount of up to about 10.0 wt. %, based on the total weight of the sanitizing composition. In other exemplary embodiments, the emulsifying agent is present in an amount of from about 0.1 to about 5.0% wt. %, or from about 0.5 to about 2.0 wt. %, based on the weight of the sanitizing composition.

The sanitizing composition may further comprise one or more deposition enhancers. A suitable deposition enhancer works unidirectionally and will allow ingredients within the composition to penetrate deeper into the stratum corneum whilst preventing the loss of materials from the skin. Advantageously, the deposition enhancer provides a cosmetically acceptable skin feel to the formulation.

In one or more embodiments, the deposition enhancers include one or more of surfactants, bile salts and derivatives thereof, chelating agents, and sulphoxides.

Some examples of acceptable deposition enhancers include hydroxypropyl methylcellulose, dimethyl sulphoxides (DMSO), DMA, DMF, 1-dodecylazacycloheptan-2-one (azone), pyrrolidones such as 2-Pyrrolidone (2P) and N-Methyl-2-Pyrrolidone (NMP), long-chain fatty acids such as oleic acid and fatty acids with a saturated alkyl chain length of about C₁₀-C₁₂, essential oils, terpenes, terpenoids, oxazolidinones such as 4-decyloxazolidin-2-one, sodium lauryl sulfate (SLS), sodium laureate, polysorbates, sodium glyacolate, sodium deoxycholate, caprylic acid, EDTA, phospholipids, C₁₂₋₁₅ Alkyl Benzoate, pentylene glycol, ethoxydiglycol, polysorbate-polyethylenesorbitan-monolaurate, and lecithin.

In one or more exemplary embodiments, the deposition enhancer is a quaternary ammonium compound such as polyquaternium-6, -7, -10, -22, -37, -39, -74 or -101.

The deposition enhancer may be included in the sanitizing composition in an amount from about 0.005 wt. % to about 10.0 wt. %, in other embodiments, from about 0.01 wt. % to about 5.0 wt. %, and in other embodiments, from about 0.05 wt. % to about 3.0 wt. %, based on the weight of the sanitizing composition.

In one or more exemplary embodiments, the deposition enhancer comprises a hydroxy-terminated polyurethane compound chosen from polyolprepolymer-2, polyolprepolymer-14, and polyolprepolymer-15. Polyolprepolymer-2 is sometimes referred to as PPG-12/SMDI copolymer. The polyurethane compound may be present in the sanitizing composition in an amount from about 0.005 wt. % to about 5.0 wt. %, in other embodiments, from about 0.01 wt. % to about 3.0 wt. %, and in other embodiments, from about 0.05 wt. % to about 1.0 wt. %, based on the weight of the sanitizing composition.

The sanitizing composition may further comprise one or more anti-irritants. Anti-irritants help reduce signs of inflammation on the skin such as swelling, tenderness, pain, itching, or redness. There are three main types of anti-irritants, all of which are envisioned as being applicable in the exemplary embodiments described herein: (1) compounds that operate by complexing the irritant itself, (2) compounds that react with the skin to block reactive sites preventing the irritant from reacting directly with the skin, and (3) compounds that prevent physical contact between the skin and irritant.

Some non-limiting examples of suitable anti-irritants include Aloe Vera, allantoin, anion-cation complexes, aryloxypropionates, azulene, carboxymethyl cellulose, cetyl alcohol, diethyl phthalate, Emcol E607, ethanolamine, glycogen, lanolin, N-(2-Hydroxylthyl) Palmitamide, N-Lauroyl Sarcosinates, Maypon 4C, mineral oils, miranols, Myristyl lactate, polypropylene glycol, polyvinyl pyrrolidone (PVP), tertiary amine oxides, thiodioglycolic acid, and zirconia. In one exemplary embodiment, the anti-irritant is avenanthrmides (avena sativa (oat), kernel oil, and glycerin) and niacinamide.

The anti-irritant may be included in the sanitizing composition in an amount up to about 10.0 wt. %, in other embodiments, from about 0.005 wt. % to about 3.0 wt. %, and in other embodiments, from about 0.01 wt. % to about 1.0 wt. %, based on the weight of the sanitizing composition.

The sanitizing composition may further comprise a wide range of optional ingredients that do not deleteriously affect the composition's ability to increase the production and/or activity of AMPs on the surface of the skin. The CTFA International Cosmetic Ingredient Dictionary and Handbook, Eleventh Edition 2005, and the 2004 CTFA International Buyer's Guide, both of which are incorporated by reference herein in their entirety, describe a wide variety of non-limiting cosmetic and pharmaceutical ingredients commonly used in the skin care industry, that are suitable for use in the compositions of the exemplary embodiments described herein. Examples of these functional classes include: abrasives, anti-acne agents, anticaking agents, antioxidants, binders, biological additives, bulking agents, chelating agents, chemical additives; colorants, cosmetic astringents, cosmetic biocides, denaturants, drug astringents, emulsifiers, external analgesics, film formers, fragrance components, opacifying agents, plasticizers, preservatives (sometimes referred to as antimicrobials), propellants, reducing agents, skin bleaching agents, skin-conditioning agents (emollient, miscellaneous, and occlusive), skin protectants, solvents, surfactants, foam boosters, hydrotropes, solubilizing agents, suspending agents (nonsurfactant), sunscreen agents, ultraviolet light absorbers, detackifiers, and viscosity increasing agents (aqueous and nonaqueous). Examples of other functional classes of materials useful herein that are well known to one of ordinary skill in the art include solubilizing agents, sequestrants, keratolytics, topical active ingredients, and the like.

In some exemplary embodiments, the sanitizing composition exhibits a pH in the range of from about 3.0 to about 12.0, or a pH in the range of from about 4.0 to about 8.0, or in the range of from about 4.5 and about 7.0. When necessary, a pH adjusting agent or constituent may be used to provide and/or maintain the pH of a composition. Exemplary pH adjusting agents include, but are not limited to, organic acids, such as citric acid, lactic acid, formic acid, acetic acid, proponic acid, butyric acid, caproic acid, oxalic acid, maleic acid, benzoic acid, carbonic acid, and the like.

The sanitizing composition may further comprise a fragrance. Any scent may be used in the sanitizing composition including, but not limited to cinnamon, clove, lavender, peppermint, rosemary, thyme, thieves, lemon, citrus, coconut, apricot, plum, watermelon, ginger and combinations thereof.

The fragrance can be included in the sanitizing composition in an amount from about 0.005 wt. % to about 5.0 wt. %, in other embodiments, from about 0.01 wt. % to about 3.0 wt. %, and in other embodiments, from about 0.05 wt. % to about 1.0 wt. %, based on the weight of the sanitizing composition. The fragrance can be any made of any perfume, essential oil, aroma compounds, fixatives, terpenes, solvents, and the like.

The form of the sanitizing composition of the exemplary embodiments described herein is not particularly limited. In some exemplary embodiments, sanitizing compositions of the exemplary embodiments described herein may be formulated as a foamable composition, a thickened gel composition, a sprayable liquid, a rinse, or may be applied to a wipe.

In some exemplary embodiments, the sanitizing composition of the exemplary embodiments described herein may be in the form of a thickened gel, with the inclusion of one or more thickeners and optionally one or more stabilizers. Examples of thickeners and stabilizers include hydroxyethyl cellulose hydroxypropyl cellulose, methyl cellulose, carboxymethyl cellulose, and ammonium acryloyldimethyltaurate/VP copolymer. Where the thickener or stabilizer is starch-based, the thickener or stabilizer may be present in an amount of up to about 10.0 wt. %, or in an amount of from about 0.1 to about 5.0 wt. %, or from about 0.2 to about 1.0 wt. %, based on the weight of the sanitizing composition. Where the thickener or stabilizer is a synthetic polymer, the thickener or stabilizer may be present in an amount of up to about 15.0 wt. %, or from about 0.05 to about 5.0 wt. %, or from about 0.1 to about 1.0 wt. %, based on the weight of the sanitizing composition.

In one or more exemplary embodiments, the sanitizing composition may be thickened with polyacrylate thickeners such as those conventionally available and/or known in the art. Examples of polyacrylate thickeners include carbomers, acrylates/C 10-30 alkyl acrylate crosspolymers (for example that sold under the trade name Carbopol® Ultrez 21 by The Lubrizol Corporation), copolymers of acrylic acid and alkyl (C₅-C₁₀) acrylate, copolymers of acrylic acid and maleic anhydride, and mixtures thereof. In one or more embodiments, the gel composition includes an effective amount of a polymeric thickener to adjust the viscosity of the gel to a viscosity range of from about 1,000 to about 65,000 centipoise (cP). In one embodiment, the viscosity of the gel is from about 5,000 to about 35,000 cP, and in another embodiment, the viscosity is from about 10,000 to about 25,000 cP. The viscosity is measured by a Brookfield RV Viscometer using RV and/or LV Spindles at 22° C.+/−3° C.

As will be appreciated by one of skill in the art, the effective amount of thickener will vary depending on a number of factors, including the amount of alcohol and other ingredients in the gel composition. In one or more embodiments, an effective amount of thickener is at least about 0.01 wt. %, based on the total weight of the gel. In other exemplary embodiments, the effective amount is at least about 0.02 wt. %, or at least about 0.05 wt. %, or at least about 0.1 wt. %, based on the total weight of the gel. In some exemplary embodiment, the effective amount of thickener is at least about 0.5 wt. %, or at least about 0.75 wt. %, based on the total weight of the gel. In one or more embodiments, the compositions according to the exemplary embodiments described herein comprise up to about 10.0 wt. % based on the total composition of a polymeric thickener. In certain embodiments, the amount of thickener is from about 0.01 to about 1.0 wt. %, or from about 0.02 to about 0.4 wt. %, or from about 0.05 to about 0.3 wt. %, based on the total weight of the gel. The amount of thickener may be from about 0.1 to about 10.0 wt. %, or from about 0.5% to about 5.0 wt. %, or from about 0.75 to about 2.0 wt. %, based on the total weight of the gel.

In one or more embodiments, the gel composition may further comprise a neutralizing agent. Examples of neutralizing agents include amines, alkanolamines, alkanolamides, inorganic bases, amino acids, including salts, esters and acyl derivatives thereof. Exemplary neutralizing agents include triethanolamine, sodium hydroxide, monoethanolamine and dimethyl stearylamine. Other neutralizing agents are also known, such as HO(CₘH₂ₘ)₂NH, where m has the value of from 2 to 3, and aminomethyl propanol, aminomethyl propanediol, and ethoxylated amines, such as PEG-25 cocamine, polyoxyethylene (5) cocamine (PEG-5 cocamine), polyoxyethylene (25) cocamine (PEG-25 cocamine), polyoxyethylene (5) octadecylamine (PEG-5 stearamine), polyoxyethylene (25) octadecylamine (PEG-25 stearamine), polyoxyethylene (5) tallowamine (PEG-5 tallowamine), polyoxyethylene (15) oleylamine (PEG-15 oleylamine), polyethylene (5) soyamine (PEG-5 soyamine), and polyoxyethylene (25) soyamine (PEG-15 soyamine). A number of these are commercially available under the trade name of Ethomeen® from Akzo Chemie America, Armak Chemicals of Chicago, Ill.

In some exemplary embodiments the neutralizing agent includes at least one of sodium hydroxide or sodium hydroxide precursors. Solutions of sodium hydroxide in water are non-limiting examples of neutralizers containing sodium hydroxide.

The neutralizing agent is employed in an effective amount to neutralize a portion of the carboxyl groups of the thickening agent, and produce the desired pH range. The pH of un-neutralized thickening agent dispersed in water is generally acidic. For example, the pH of Carbopol® polymer dispersions is approximately in the range of 2.5 to 3.5, depending on the polymer concentration. An effective amount of neutralizing agent, when added to the thickener dispersion, adjusts the pH to a desired range of about 4.1 to 4.8, or of about 4.2 to 4.6. The amount of neutralizing agent necessary to effect this pH range will vary depending on factors such as the type of thickening agent, the amount of thickening agent, etc. However, in general, amounts less than 1.0% by weight and ranging from about 0.001 to about 0.3 wt. %, based on the weight of the sanitizing composition, of the neutralizing agent are considered sufficient and effective.

In one or more embodiments, the sanitizing composition is formulated as a foamable composition. One or more foam agents may optionally be included in the foamable composition.

Any foaming agent conventionally known and used may be employed in the sanitizing composition. In one or more embodiments, the foam agent comprises a non-ionic foam agent such as decyl glucoside or an amphoteric foam agent such as cocamidopropylbetaine. In one or more embodiments, the amount of nonionic or amphoteric foam agent is from about 0.5 to about 3.5 wt. %, in other embodiments from about 1.0 to about 3.0 wt. %, based on the weight of the sanitizing composition. In one or more embodiments, the amount of decyl glucoside or cocamidopropylbetaine is from about 0.5 to about 3.5 wt. %, in other embodiments from about 1.0 to about 3.0 wt. %, based on the weight of the sanitizing composition.

In some exemplary embodiments, the foaming agents include one or more of silicone glycol and fluorosurfactants. Silicone glycols may be generally characterized by containing one or more Si—O—Si linkages in the polymer backbone. Silicone glycols include organopolysiloxane dimethicone polyols, silicone carbinol fluids, silicone polyethers, alkylmethyl siloxanes, amodimethicones, trisiloxane ethoxylates, dimethiconols, quaternized silicone glycols, polysilicones, silicone crosspolymers, and silicone waxes.

Examples of silicone glycols include dimethicone PEG-7 undecylenate, PEG-10 dimethicone, PEG-8 dimethicone, PEG-12 dimethicone, perfluorononylethyl carboxydecal PEG 10, PEG-20/PPG-23 dimethicone, PEG-11 methyl ether dimethicone, bis-PEG/PPG-20/20 dimethicone, silicone quats, PEG-9 dimethicone, PPG-12 dimethicone, fluoro PEG-8 dimethicone, PEG-23/PPG-6 dimethicone, PEG-20/PPG-23 dimethicone, PEG 17 dimethicone, PEG-5/PPG-3 methicone, bis-PEG-18 methyl ether dimethyl silane, bis-PEG-20 dimethicone, PEG/PPG-20/15 dimethicone copolyol and sulfosuccinate blends, PEG-8 dimethicone\dimmer acid blends, PEG-8 dimethicone\fatty acid blends, PEG-8 dimethicone\cold pressed vegetable oil\polyquaternium blends, random block polymers and mixtures thereof.

The amount of silicone glycol foam agent is not particularly limited, so long as an effective amount to produce foaming is present. In certain embodiments, the effective amount to produce foaming may vary, depending on the amount of alcohol and other ingredients that are present. In one or more embodiments, the composition includes at least about 0.002 wt. % of silicone glycol foam agent, based on the weight of the sanitizing composition. In another embodiment, the composition includes at least about 0.01 wt. % of silicone glycol foam agent, based on the weight of the sanitizing composition. In yet another embodiment, the sanitizing composition includes at least about 0.05 wt. % of silicone glycol foam agent, based on the weight of the sanitizing composition. In a preferred embodiment the foam agent is Peg-12 dimethicone.

In some exemplary embodiments, the foam agent is present in an amount of from about 0.002 to about 4.0 wt. %, or in an amount of from about 0.01 to about 2.0 wt. %, based on the weight of the sanitizing composition. In a preferred embodiment the foam agent is present in an amount of about 1.0 to 1.8 wt. %, based on the weight of the sanitizing composition. It is envisioned that higher amounts may also be effective to produce foam. All such weights as they pertain to listed ingredients are based on the active level, and therefore, do not include carriers or by-products that may be included in commercially available materials, unless otherwise specified.

In other embodiments, it may be desirable to use higher amounts of foam agent. For example, in certain embodiments where the foaming composition of the exemplary embodiments described herein includes a cleansing or sanitizing product that is applied to a surface and then rinsed off, higher amounts of foam agent may be employed. In these embodiments, the amount of foam agent is present in amounts up to about 35.0 wt. %, based on the weight of the sanitizing composition.

The sanitizing composition of the exemplary embodiments described herein may be formulated as an aerosol or non-aerosol foamable composition. In some exemplary embodiments the sanitizing composition is dispensed from an unpressurized or low-pressure dispenser which mixes the composition with air.

In some exemplary embodiments, the viscosity of the non-aerosol foamable composition is less than about 100 mPas, in one embodiment less than about 50 mPas, and in another embodiment less than about 25 mPas.

The sanitizing composition according to the exemplary embodiments described herein may be employed in any type of dispenser typically used for gel products, for example pump dispensers. A wide variety of pump dispensers are suitable. Pump dispensers may be affixed to bottles or other free-standing containers. Pump dispensers may be incorporated into wall-mounted dispensers. Pump dispensers may be activated manually by hand or foot pump, or may be automatically activated. Useful dispensers include those available from GOJO Industries under the designations NXT®, TFX™, DPX™, FMX™, ADX™, LTX™, and CXT™ as well as traditional bag-in-box dispensers. Examples of dispensers are described in U.S. Pat. Nos. 5,265,772, 5,944,227, 6,877,642, 7,028,861, 7,611,030, 7,621,426, 8,740,019, 8,991,657, 9,027,790, 9,073,685, 9,101,250, and 9,204,767, all of which are incorporated herein by reference. In one or more embodiments, the dispenser includes an outlet such as a nozzle, through which the composition is dispensed. In some exemplary embodiments, the sanitizing composition is used in dispensers that employ foaming pumps, which combine ambient air or an inert gas and the composition in a mixing chamber and pass the mixture through a mesh screen.

In one or more embodiments, the sanitizing composition is integrated into wipe composition. Wipe compositions in accordance with the exemplary embodiments described herein include at least one alcohol, a C₁₋₁₀ alkanediol enhancer, and are applied to a wipe substrate.

Wipe substrates used in antimicrobial wipes are further described in U.S. Pat. Nos. 5,686,088, 6,410,499, 6,436,892, 6,495,508, 6,844,308, 9,096,821, which are incorporated herein by reference. In one or more embodiments, the wipe may comprise a laminate formed by spunbonding/meltblowing/spunbonding (SMS). Generally, an SMS material contains a meltblown web sandwiched between two exteriors spunbond webs. SMS materials are further described in U.S. Pat. Nos. 4,041,203, 5,169,706, 5,464,688, and 4,766,029, and are commercially available, for example from Kimberly-Clark Corporation under marks such as Spunguard 7 and Evolution 7. The SMS laminate may be treated or untreated.

In other exemplary embodiments, the sanitizing composition is formulated as a spray. In some exemplary embodiments, the spray is a nasal spray that is designed to be used in the nose. The particular delivery method of the nasal spray is not particularly limited. Exemplary delivery methods and base compositions which are envisioned as suitable for the exemplary embodiments disclosed herein are disclosed in U.S. Pat. Nos. 8,053,005; 8,158,163; 8,778,415; 8,999,406; and 9,463,212, all to Global Life Technologies Corp., all of which are incorporated herein by reference. In some exemplary embodiments, the nasal spray is used in a hospital to treat some or all admitted patients. In some exemplary embodiments, hospital and out-patient healthcare employees are treated with the nasal spray. In some exemplary embodiments, patients with a history or recurrent folliculitis, furunculosis, and/or staph aureus are treated with the nasal spray. In still other exemplary embodiment, family members of patients suffering from these conditions are treated with the nasal spray.

In some exemplary embodiments, the nasal spray sanitizing composition comprises one or more C₁₋₈ alcohols and an active ingredient. In some exemplary embodiments, the active ingredient is one or more of an extract and a polypeptide. In some exemplary embodiments, the nasal spray sanitizing composition comprises various fragrances. In some exemplary embodiments, application of the nasal spray reduces pathogen binding in the nose by an amount that is statistically significant, as compared to an otherwise identical nasal spray without the active ingredient.

In some exemplary embodiments, the nasal spray kills staph bacteria, such as that associated with and responsible for methicillin-resistant staphylococcus aureus (MRSA). In some exemplary embodiments, the nasal spray with alcohol and active ingredient helps to reestablish the healthy normal nasal flora biome. In some exemplary embodiments, the nasal spray provides efficacy against pathogenic bacteria and does not promote bacterial-resistance developing even with extended use. In some exemplary embodiments, the nasal spray helps to fight and defend agaisnt pathogens associated with of clostridium difficile infections of the intestine, C. difficile, MRSA, folliculitis, furunculosis, and staph aureus.

In some exemplary embodiments, a sanitizing composition comprising up to about 15.0 wt. % of a polypeptide active ingredient increases the production and/or activity of defensins, such as HBD-1 by a statistically significant amount, as compared to an otherwise identical composition that does not include the active ingredient. In some exemplary embodiments, a sanitizing composition comprising up to about 15.0 wt. % of a polypeptide active ingredient increases the production of defensins, such as HBD-1 by at least 25%, or at least 100%, or at least 500%, or at least 800%, or at least 1000%, as compared to an otherwise identical composition that does not include the active ingredient. In some exemplary embodiments, a sanitizing composition comprising up to about 15.0 wt. % of a polypeptide active ingredient increases the production/activity of defensins, such as HBD-1 by at least 1,400%, or by at least 1,700%, as compared to an otherwise identical sanitizing composition that does not include the active ingredient.

In some exemplary embodiments, a sanitizing composition comprising up to about 15.0 wt. % of a polypeptide active ingredient increases the production and/or activity of defensins, such as HBD-2 by a statistically significant amount, as compared to an otherwise identical sanitizing composition that does not include the active ingredient. In some exemplary embodiments, a sanitizing composition comprising up to about 15.0 wt. % of a polypeptide active ingredient increases the production of defensins, such as HBD-2 by at least 25%, or at least 100%, or at least 500%, or at least 800%, or at least 1000%, as compared to an otherwise identical sanitizing composition that does not include the active ingredient. In some exemplary embodiments, a sanitizing composition comprising up to about 15.0 wt. % of a polypeptide active ingredient increases the production/activity of defensins, such as HBD-2 by at least 1,100%, or by at least 1,200%, or by at least 2,000%, as compared to an otherwise identical sanitizing composition that does not include the active ingredient.

In some exemplary embodiments, a sanitizing composition comprising up to about 15.0 wt. % of a polypeptide active ingredient increases the production and/or activity of defnsins, such as HBD-3 by a statistically significant amount, as compared to an otherwise identical sanitizing composition that does not include the active ingredient. In some exemplary embodiments, a sanitizing composition comprising up to about 15.0 wt. % of an active ingredient increases the production of defensins, such as HBD-3 by at least 25%, or at least 50%, or at least 100%, or at least 500%, or at least 800%, or at least 1000%, as compared to an otherwise identical sanitizing composition that does not include the active ingredient. In some exemplary embodiments, a sanitizing composition comprising up to about 15.0 wt. % of a polypeptide active ingredient increases the production/activity of defensins such as HBD-3 by at least 2,000%, or by at least 2,500%, or by at least 4,000%, as compared to an otherwise identical sanitizing composition that does not include the active ingredient.

In some exemplary embodiments, a sanitizing composition comprising up to about 15.0 wt. % of an extract active ingredient increases the production and/or activity of defensins, such as HBD-1 by a statistically significant amount, as compared to an otherwise identical sanitizing composition that does not include the active ingredient. Particularly, a sanitizing composition comprising up to about 15.0 wt. % of a hydrolysate of linseed proteins increases the production/activity of defensins, such as HBD-1 by at least 10%, or at least 20%, or at least 50%, or at least 75%, or at least 95%, as compared to an otherwise identical sanitizing composition that does not include the active ingredient.

In some exemplary embodiments, a sanitizing composition comprising up to about 15.0 wt. % of an extract active ingredient increases the production and or/activity of defensins, such as HBD-2 by a statistically significant amount, as compared to an otherwise identical sanitizing composition that does not include the active ingredient. Particularly, a sanitizing composition comprising up to about 15.0 wt. % of a hydrolysate of linseed proteins increases the production/activity of defenins, such as HBD-2 by at least 5%, or at least 10%, or at least 20%, or at least 23%, as compared to an otherwise identical sanitizing composition that does not include the active ingredient

In some exemplary embodiments, a sanitizing composition comprising up to about 15.0 wt. % of an extract active ingredient increases the production and/or activity of defensins, such as HBD-3 by a statistically significant amount, as compared to an otherwise identical sanitizing composition that does not include the active ingredient. Particularly, a sanitizing composition comprising up to about 15.0 wt. % of a hydrolysate of linseed proteins increases the production/activity of defensins, such as HBD-3 by at least 5%, or at least 10%, or at least 20%, or at least 29%, as compared to an otherwise identical sanitizing composition that does not include the active ingredient.

In some exemplary embodiments, a sanitizing composition comprising up to about 15.0 wt. % of an extract active ingredient increases the production and/or activity of cathelicidin-related AMPS, such as LL-37 by a statistically significant amount, as compared to an otherwise identical sanitizing composition that does not include the active ingredient. Particularly, a sanitizing composition comprising up to about 15.0 wt. % of a hydrolysate of linseed proteins increases the production/activity of cathelicidin-related AMPS, such as LL-37 by at least 5%, or at least 10%, or at least 20%, or at least 30%, or at least 38%, as compared to an otherwise identical sanitizing composition that does not include the active ingredient.

In some exemplary embodiments, a sanitizing composition comprising up to about 15.0 wt. % of an extract active ingredient decreases the production and/or activity of pro-inflammatory factors, such as IL-8 by a statistically significant amount, as compared to an otherwise identical sanitizing composition that does not include the active ingredient. Particularly, a sanitizing composition comprising up to about 15.0 wt. % of a hydrolysate of linseed proteins decreases the production/activity of pro-inflammatory factors, such as IL-8 by at least 5%, or at least 10%, or at least 20%, or at least 30%, or at least 33%, as compared to an otherwise identical sanitizing composition that does not include the active ingredient.

In some exemplary embodiments, a sanitizing composition comprising up to about 15.0 wt. % of an extract active ingredient in a leave-on formulation increases the production and/or activity of defensins, such as HBD-1 by a statistically significant amount, as compared to an otherwise identical sanitizing composition that does not include the active ingredient. Particularly, a sanitizing composition comprising up to about 15.0 wt. % of a hydrolysate of linseed proteins in a leave-on formulation increases the production/activity of defensins, such as HBD-1 by at least 1 pg/mL, or at least 6 pg/mL, or at least 8 ng/mL, or at least 10 pg/mL, or at least 16 pg/mL, as compared to an otherwise identical sanitizing composition that does not include the active ingredient.

In some exemplary embodiments, a sanitizing composition comprising up to about 15.0 wt. % of an extract active ingredient in a leave-on formulation increases the production and/or activity of defensins, such as HBD-2 by a statistically significant amount, as compared to an otherwise identical sanitizing composition that does not include the active ingredient. Particularly, a sanitizing composition comprising up to about 15.0 wt. % of a hydrolysate of linseed proteins in a leave-on formulation increases the production/activity of defenins, such as HBD-2 by at least 1 pg/mL, or at least 4 pg/mL, or at least 10 pg/mL, or at least 15 pg/mL, or at least 20 pg/mL, as compared to an otherwise identical sanitizing composition that does not include the active ingredient.

In some exemplary embodiments, a sanitizing composition comprising up to about 15.0 wt. % of an extract active ingredient in a leave-on formulation increases the production and/or activity of defensins, such as HBD-3 by a statistically significant amount, as compared to an otherwise identical sanitizing composition that does not include the active ingredient. Particularly, a sanitizing composition comprising up to about 15.0 wt. % of a hydrolysate of linseed proteins in a leave-on formulation increases the production/activity of defensins, such as HBD-3 by at least at least 1 pg/mL, as compared to an otherwise identical sanitizing composition that does not include the active ingredient.

### EXAMPLES

The following examples are included for purposes of illustration and are not intended to limit the scope of the methods described herein.

### Example 1

To determine the optimal dose of active ingredient, test dose response studies were run using both Decorinyl® and Pamitoyl Pentapeptided-3. These test dose response studies were commissioned to determine the concentration of HBD-1 at various levels of the active ingredients. Neonatal Human Epidermal Keratinocytes (NHEK; Life Technology, Grand Island, N.Y., USA) were cultured with keratinocyte growth medium (KGM, Medium 154: M-154-500 Life Technology with supplements S-001, Life Technologies). NHEK were seeded into 96-well plates at a density of 10000 cells in 200 μl medium per well. After 48 hours, the cells were incubated with varying concentrations of each ingredient solution in a culture medium (KGM) overnight (16 hours) at 37° C., 5% CO₂ and 95% humidity at four replicates for each concentration. Each of these active ingredients was tested at the following weight percents based on the weight of the total culture: 0.02 wt. %, 0.05 wt. %, 0.1 wt. %, 0.2 wt. %, 0.5 wt. %, 1.0 wt. %, 2.0 wt. %. Each of these compositions was compared to a control culture medium.

HBD-1 was detected using HBD-1 ELISA (enzyme-linked immunosorbent assay) developing kits (commercially available from Peprotech). ELISA were performed according to the manufactory instructions of each kit by adding 100 μl/well of culture medium after overnight treatment. The substrate of ELISA reaction was using the substrate reagent from R&D Systems (DY999), and the reactions were stopped by adding 50 μl of 1N H₂SO₄ in each well. The results were measured using a colorimeter, absorbance was measured at 450 nanometers (nm) within 30 minutes. Wavelength correction was set to 570 nm. The concentration of each sample was calculated using ELISA standard curve.

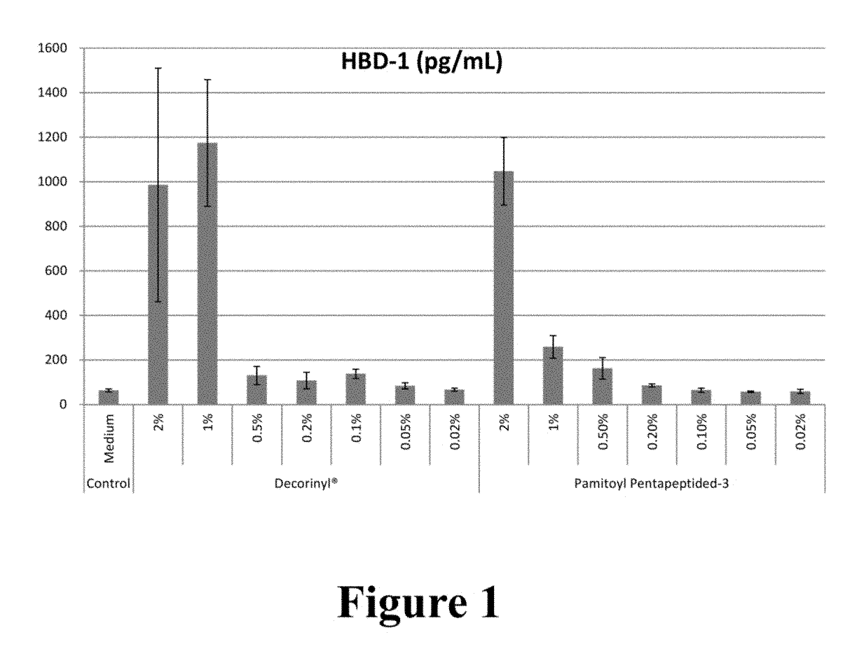

The results are listed below in Table 1 and depicted graphically in FIG. 1. As illustrated below, a 1.0 and 2.0 wt. % concentration of Decorinyl® demonstrated an increase in HBD-1 production and/or activity of 1763% and 1465% were observed for 1.0 wt. % and 2.0 wt. % Decorinyl®, respectively. Increases in HBD-1 production and/or activity of 311% and 1561% were observed for 1.0 wt. % and 2.0 wt. % Pamitoyl Pentapeptided-3, respectively.

TABLE 1

| Active Ingredient | wt. % | HBD-1 (pg/mL) |
|---|---|---|
| Control | Medium | 63 |
| Decorinyl ® | 2% | 986 |
| | 1% | 1174 |
| | 0.5% | 130 |
| | 0.2% | 107 |
| | 0.1% | 138 |
| | 0.05% | 84 |
| | 0.02% | 67 |
| Pamitoyl Pentapeptided-3 | 2% | 1047 |
| | 1% | 259 |
| | 0.50% | 162 |
| | 0.20% | 85 |
| | 0.10% | 64 |
| | 0.05% | 57 |
| | 0.02% | 59 |

### Example 2

To determine the optimal dose of active ingredient, test dose response studies were run using both Decorinyl® and Pamitoyl Pentapeptided-3. These test dose response studies were commissioned to determine the concentration of HBD-2 at various levels of the active ingredients. Neonatal Human Epidermal Keratinocytes (NHEK; Life Technology, Grand Island, N.Y., USA) were cultured with keratinocyte growth medium (KGM, Medium 154: M-154-500 Life Technology with supplements S-001, Life Technologies). NHEK were seeded into 96-well plates at a density of 10000 cells in 200 μl medium per well. After 48 hours, the cells were incubated with varying concentrations of each ingredient solution in a culture medium (KGM) overnight (16 hours) at 37° C., 5% CO₂ and 95% humidity at four replicates for each concentration. Each of these active ingredients were tested at the following weight percents based on the weight of the total culture: 0.02 wt. %, 0.05 wt. %, 0.1 wt. %, 0.2 wt. %, 0.5 wt. %, 1.0 wt. %, 2.0 wt. %. Each of these compositions was compared to a control culture medium.

HBD-2 was detected using HBD-2 ELISA developing kits (commercially available from Peprotech). ELISA were performed according to the manufactory instructions of each kit by adding 100 μl/well of culture medium after overnight treatment. The substrate of ELISA reaction was using the substrate reagent from R&D Systems (DY999), and the reactions were stopped by adding 50 μl of 1N H₂SO₄ in each well. The results were measured using a colorimeter, absorbance was measured at 450 nanometers (nm) within 30 minutes. Wavelength correction was set to 570 nm. The concentration of each sample was calculated using ELISA standard curve.

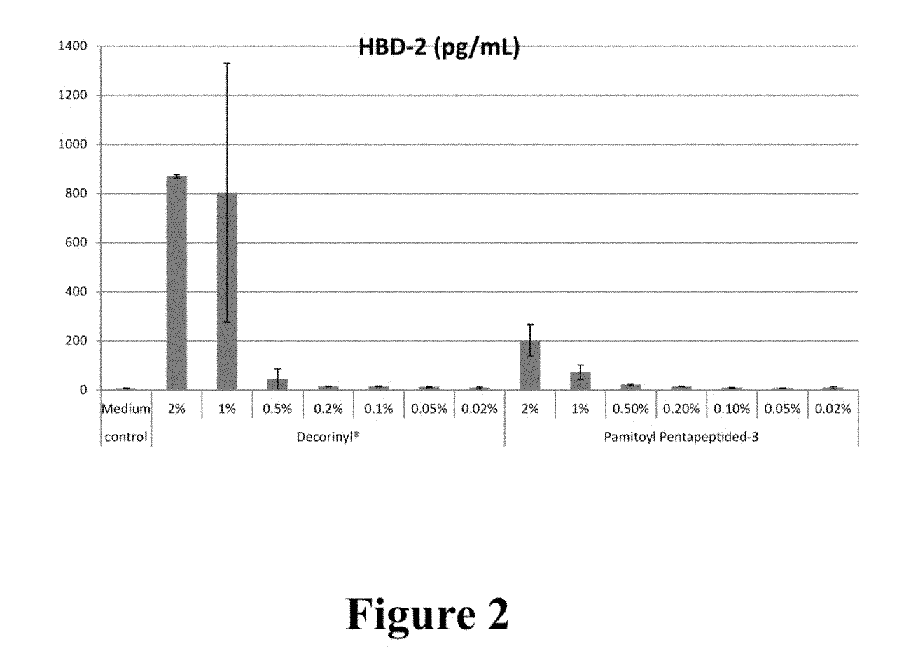

The results are listed below in Table 2 and depicted graphically in FIG. 2. Increases in HBD-2 production and/or activity of 11,371% and 12,329% were observed for 1.0 wt. % and 2.0 wt. % Decorinyl® respectively. An increase in HBD-2 production and/or activity of 2800% was observed for 2.0 wt. % Pamitoyl Pentapeptided-3.

TABLE 2

| Active Ingredient | wt. % | HBD-2 (pg/mL) |
|---|---|---|
| Control | Medium | 7 |
| Decorinyl ® | 2% | 870 |
| | 1% | 803 |
| | 0.5% | 44 |
| | 0.2% | 15 |
| | 0.1% | 15 |
| | 0.05% | 12 |
| | 0.02% | 9 |
| Pamitoyl Pentapeptided-3 | 2% | 203 |
| | 1% | 72 |
| | 0.50% | 21 |
| | 0.20% | 14 |
| | 0.10% | 9 |
| | 0.05% | 8 |
| | 0.02% | 9 |

### Example 3

To determine the optimal dose of active ingredient, test dose response studies were run using both Decorinyl® and Pamitoyl Pentapeptided-3. These test dose response studies were commissioned to determine the concentration of HBD-3 at various levels of the active ingredients. Neonatal Human Epidermal Keratinocytes (NHEK; Life Technology, Grand Island, N.Y., USA) were cultured with keratinocyte growth medium (KGM, Medium 154: M-154-500 Life Technology with supplements S-001, Life Technologies). NHEK were seeded into 96-well plates at a density of 10000 cells in 200 μl medium per well. After 48 hours, the cells were incubated with varying concentrations of each ingredient solution in a culture medium (KGM) overnight (16 hours) at 37° C., 5% CO₂ and 95% humidity at four replicates for each concentration. Each of these active ingredients was tested at the following weight percents based on the weight of the total culture: 0.02 wt. %, 0.05 wt. %, 0.1 wt. %, 0.2 wt. %, 0.5 wt. %, 1.0 wt. %, 2.0 wt. %. Each of these compositions was compared to a control culture medium.

HBD-3 was detected using HBD-3 ELISA developing kits (commercially available from Peprotech). ELISA were performed according to the manufactory instructions of each kit by adding 100 μl/well of culture medium after overnight treatment. The substrate of ELISA reaction was using the substrate reagent from R&D Systems (DY999), and the reactions were stopped by adding 50 μl of 1N H₂SO₄ in each well. The results were measured using a colorimeter, absorbance was measured at 450 nanometers (nm) within 30 minutes. Wavelength correction was set to 570 nm. The concentration of each sample was calculated using ELISA standard curve.

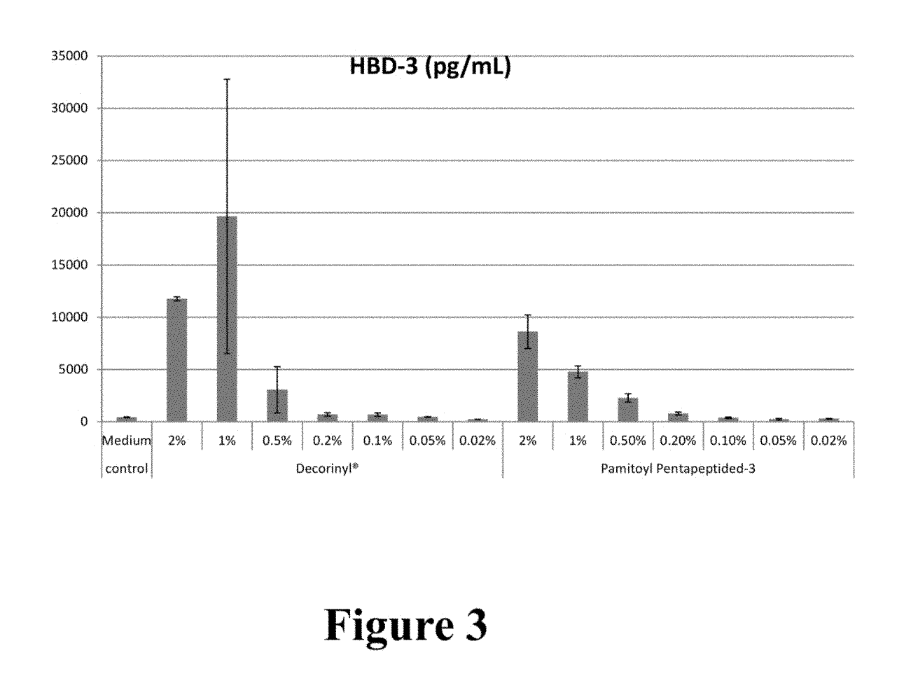

The results are shown below in Table 3 and depicted graphically in FIG. 3. Increases in HBD-3 production and/or activity of 4438% and 2616% were observed for 1.0 wt. % and 2.0 wt. % Decorinyl® respectively. Increases in HBD-3 production and/or activity of 1005% and 1890% were observed for 1.0 wt. % and 2.0 wt. % Pamitoyl Pentapeptided-3, respectively.

TABLE 3

| Active Ingredient | wt. % | HBD-3 (pg/mL) |
|---|---|---|
| Decorinyl ® | 2% | 11759 |
| | 1% | 19652 |
| | 0.5% | 3058 |
| | 0.2% | 703 |
| | 0.1% | 682 |
| | 0.05% | 456 |
| | 0.02% | 226 |
| Pamitoyl Pentapeptided-3 | 2% | 8617 |
| | 1% | 4783 |
| | 0.50% | 2278 |
| | 0.20% | 775 |
| | 0.10% | 387 |
| | 0.05% | 242 |
| | 0.02% | 288 |

### Example 4

Lipigenine™ was tested for its ability to stimulate an increase in HBD-1 concentration. The HBD-1 standard ABTS (2,2′-Azinobis [3-ethylbenzothiazoline-6-sulfonic acid]-diammonium salt) ELISA development kits were obtained from PeproTech (Cat#900-K202). ELISA were performed according to the manufactory instructions of each kit by adding 100 μl/well of culture medium after overnight treatment. The substrate of ELISA reaction was using the substrate reagent from R&D Systems (DY999), and the reactions were stopped by adding 50 μl of 1N H₂SO₄ in each well. The Lipigenine™ culture was compared to the control medium which contained no other ingredients. The results were measured using a colorimeter, absorbance was measured at 450 nanometers (nm) within 30 minutes. Wavelength correction was set to 570 nm. The concentration of each sample was calculated using ELISA standard curve.

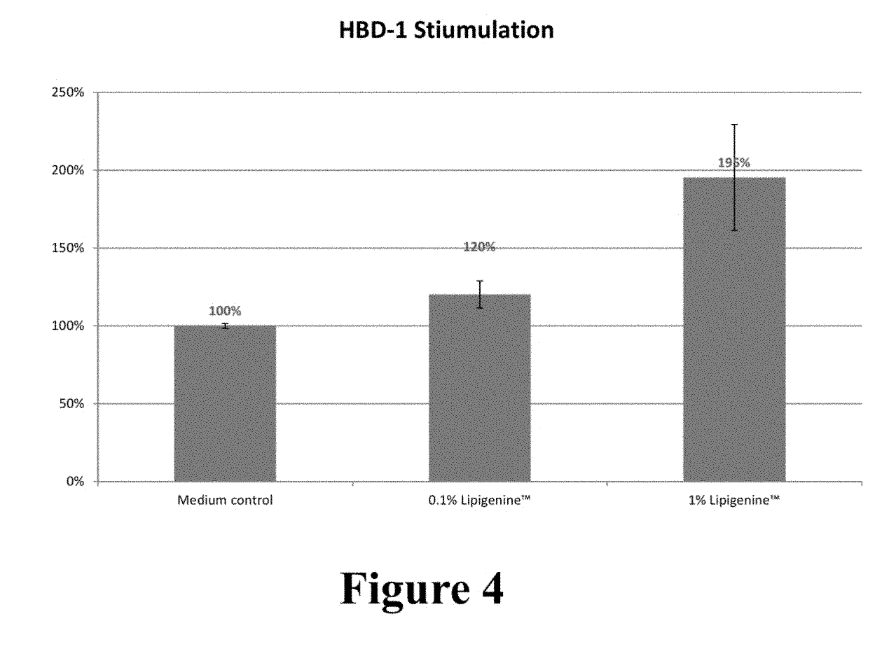

The addition of Lipigenine™ showed high HBD-1 production and/or activity at both 0.1% and 1% Lipigenine™ in solution as compared to the control. An increase in HBD-1 production and/or activity of 20% was observed for 0.1% Lipigenine™ while an increase in HBD-1 production and/or activity of 95% was observed for 1% Lipigenine™. These results are shown in FIG. 4.

### Example 5

Lipigenine™ was tested for its ability to stimulate an increase in HBD-2 production and/or activity. The HBD-2 standard ABTS ELISA development kits were obtained from PeproTech (Cat#900-K172). ELISA was performed according to the manufactory instructions of each kit by adding 100 μl/well of culture medium after overnight treatment. The substrate of ELISA reaction was using the substrate reagent from R&D Systems (DY999), and the reactions were stopped by adding 50 μl of 1N H₂SO₄ in each well. The Lipigenine™ culture was compared to the control medium which contained no other ingredients. The results were measured using a colorimeter, absorbance was measured at 450 nanometers (nm) within 30 minutes. Wavelength correction was set to 570 nm. The concentration of each sample was calculated using ELISA standard curve.

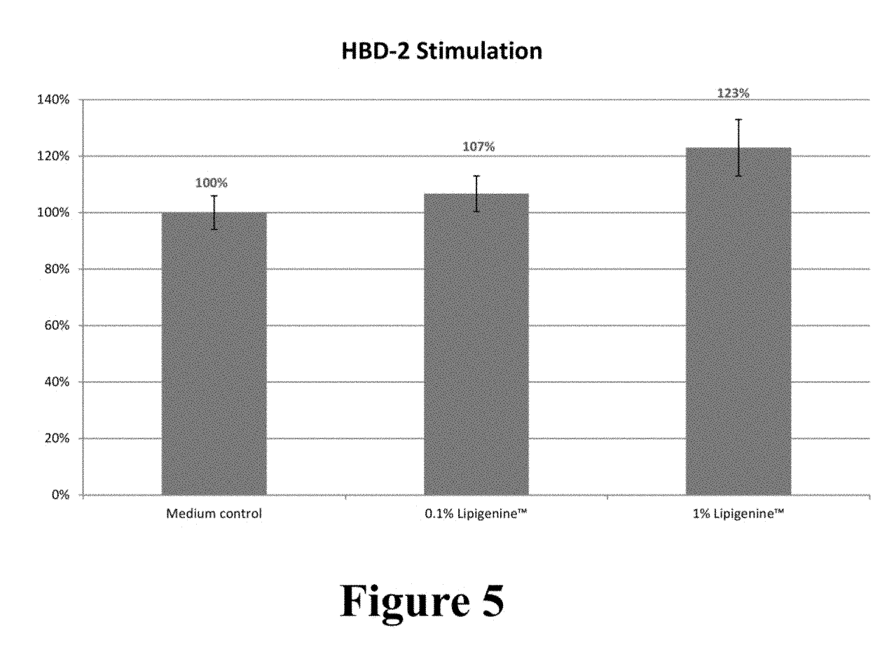

The addition of Lipigenine™ showed increased HBD-2 production and/or activity at both 0.1% and 1% Lipigenine™ in solution as compared to the control. An increase in HBD-2 production and/or activity of 7% was observed for a 0.1% Lipigenine™ formulation while an increase in HBD-2 production and/or activity of 23% was observed for a 1% Lipigenine™ formulation. These results are shown in FIG. 5.

### Example 6

Lipigenine™ was tested for its ability to stimulate an increase in HBD-3 production and/or activity. The HBD-3 standard ABTS ELISA development kit was obtained from PeproTech (Cat#900-K210). ELISA were performed according to the manufactory instructions of each kit by adding 100 μl/well of culture medium after overnight treatment. The substrate of ELISA reaction was using the substrate reagent from R&D Systems (DY999), and the reactions were stopped by adding 50 μl of 1N H₂SO₄ in each well. The Lipigenine™ culture was compared to the control medium which contained no other ingredients. The results were measured using a colorimeter, absorbance was measured at 450 nanometers (nm) within 30 minutes. Wavelength correction was set to 570 nm. The concentration of each sample was calculated using ELISA standard curve.

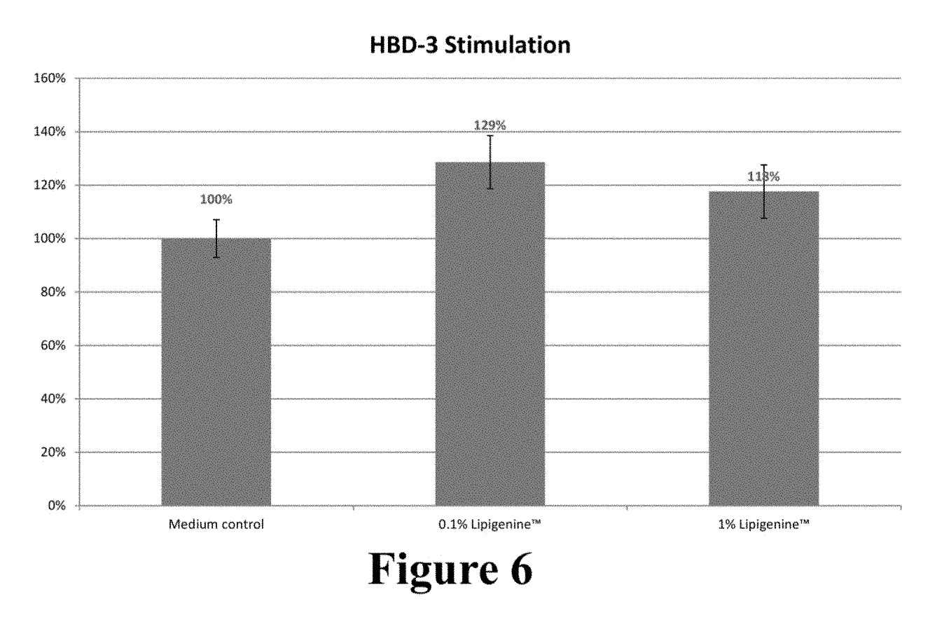

The addition of Lipigenine™ showed increased HBD-3 production and/or activity at both 0.1% and 1% Lipigenine™ in solution as compared to the control. An increase in HBD-3 concentration of 29% was observed for a 0.1% Lipigenine™ formulation while an increase in HBD-3 production and/or activity of 18% was observed for a 1% Lipigenine™ formulation. These results are shown in FIG. 6.

### Example 7

A sanitizing composition with Lipigenine™ was tested for its ability to increase production and/or activity of Cathelicidins (such as LL37), an amphipathic alpha-helical peptide that plays an important role in defense against local infection and invasion of pathogens at sites of inflammation and wounds. The human LL-37 ELISA kit was obtained from Hycult Biotech (Cat#HK321). ELISA were performed according to the manufactory instructions of each kit by adding 100 μl/well of culture medium after overnight treatment. The results were measured using a colorimeter, absorbance was measured at 450 nanometers (nm) within 30 minutes. Wavelength correction was set to 570 nm.

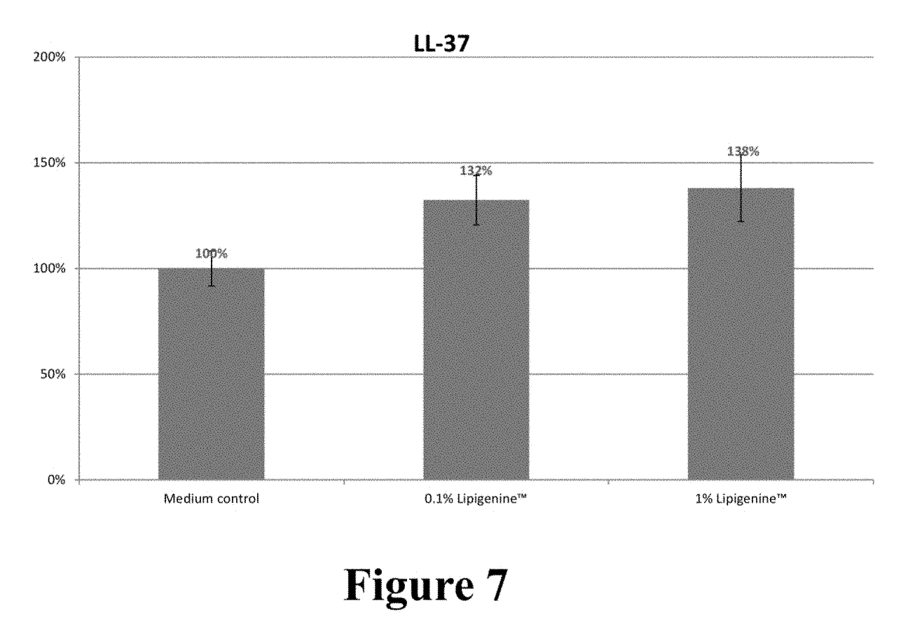

The addition of Lipigenine™ showed increased LL-37 production and/or activity at both 0.1% and 1% Lipigenine™ in solution as compared to the control. An increase in LL-37 production and/or activity of 32% for a 0.1% Lipigenine™ formulation while an increase in LL-37 production and/or activity of 38% was observed for a 1% Lipigenine™ formulation. These results are shown in FIG. 7.

### Example 8

A sanitizing composition with Lipigenine™ was tested for its ability to decrease production and/or activity of Interleukin 8 (IL-8 or CXCL8) which is a chemokine and proinflammatory cytokine produced by macrophages and other cell types such as epithelial cells. It is secreted from keratinocytes in skin in response to inflammatory stimuli. IL-8 is secreted and is an important mediator of the immune reaction in the innate immune system response. IL-8 over-expressed is a biomarker of skin irritation. IL-8 is associated with inflammation and plays a role in colorectal cancer.

For Control A, human dermal keratinocytes were left untreated. No irritation is expected, and therefore Control A provides a baseline (set as 0). For Control B, IL-8 is induced in human dermal keratinocytes by applying a surfactant mixture that is a combination of sodium laureth sulfate and polyquaternium-10 (set as 100%). For all other samples, the human dermal keratinocytes are co-treated with the surfactant mixture and a composition containing indicated concentration of Lipigenine™. Decreased IL-8 production and/or activity reflects an ingredient's anti-irritation activity. In order to carry out the test method, an assay kit was employed that was obtained from R&D Systems: Human CXCL8/IL-8 Duoset ELISA Kit (DY208). ELISA was performed after overnight treatment using by applying 100 μl/well of culture medium according to the manufactory instruction of the ELISA kit. The results were measured using a colorimeter, absorbance was measured at 450 nanometers (nm) within 30 minutes. Wavelength correction was set to 570 nm.

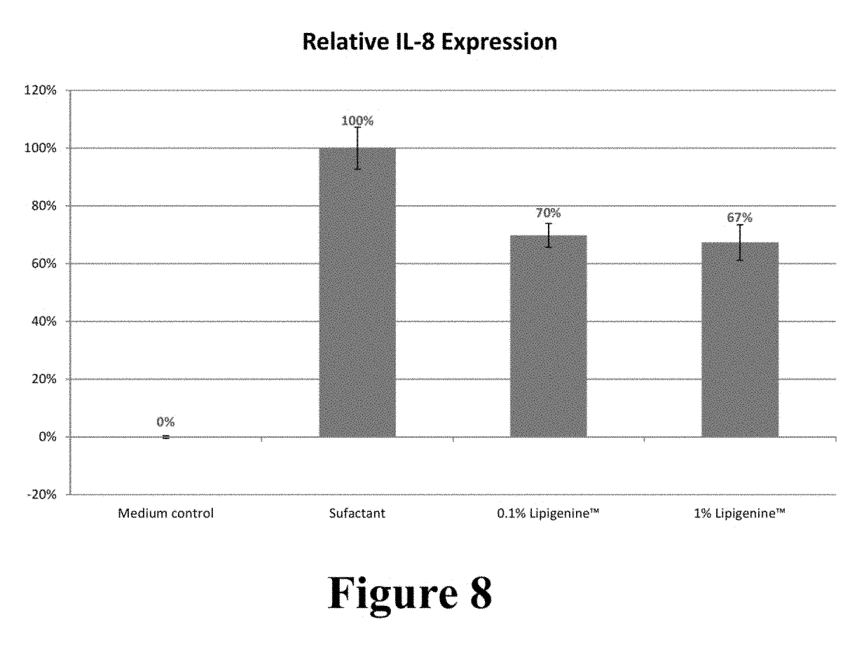

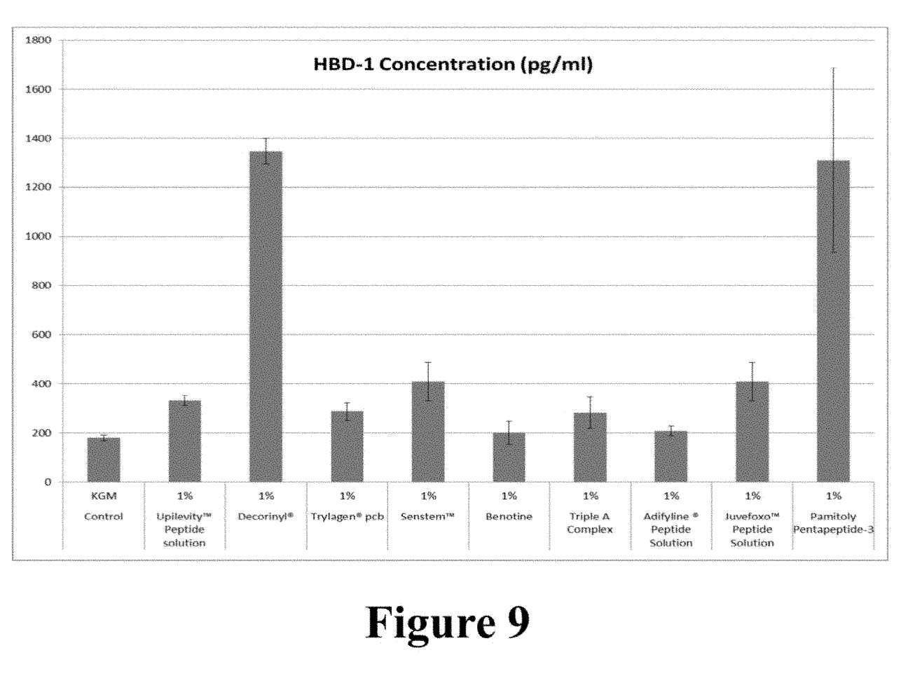

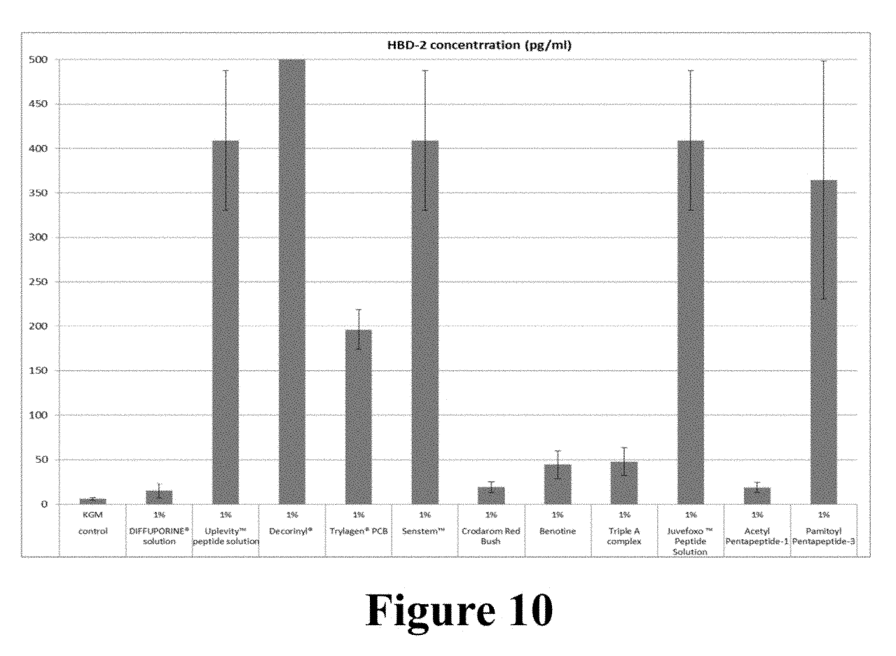

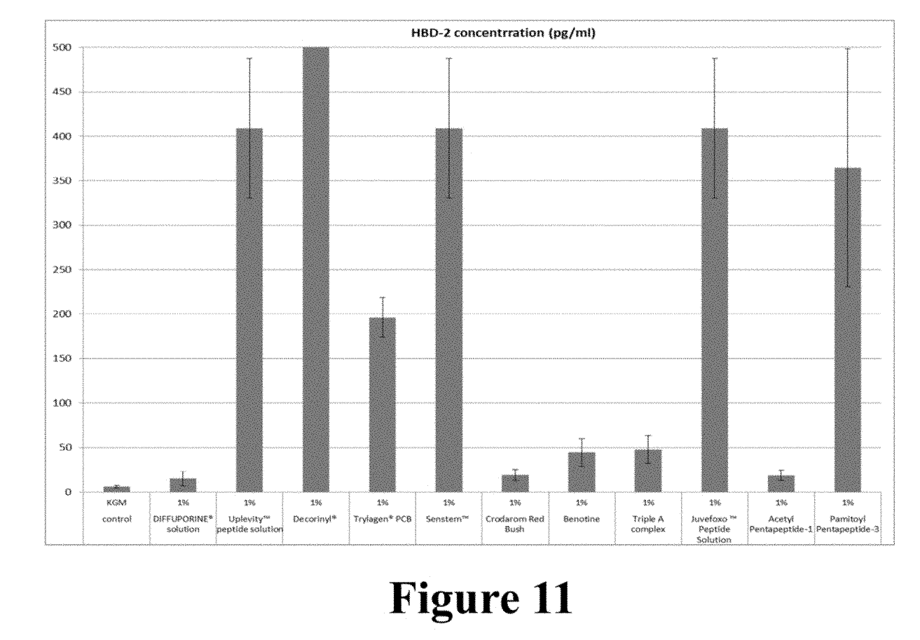

The addition of Lipigenine™ showed reduced IL-8 production and/or activity at both 0.1% and 1% Lipigenine™ in solution as compared to a surfactant. A decrease in IL-8 production and/or activity of 30% was observed for a 0.1% Lipigenine™ formulation while a decrease in IL-8 production and/or activity of 33% was observed for a 1% Lipigenine™ formulation. These results are shown in FIG. 8.

### Example 9

Tape stripping tests were also performed with 1% Lipigenine™ in a hand sanitizer gel formulation (leave-on) to determine the production and/or activity of AMPs including HBD-1, HBD-2, and HBD-3 on the skin as compared to a hand sanitizer formulation gel without Lipigenine™ (leave-on). 7 layers of tape strips were applied to the skin at two adjacent sites for both the hand sanitizer with Lipigenine™ and the hand sanitizer without Lipigenine™. The strips were applied after the two formulations had been used to clean each skin site. After application, the first layer of tape was discarded. Thereafter, layers 2-4 were combined (the “Upper Layers”) and layers 5-7 were combined (the “Lower Layers”). These tape striping experiments were run at 0 days (before application), 5 days after application, and 10 days after application to observe increases in AMP concentration over time. Each of the Upper Layers and the Lower Layers were placed in a glass vial and frozen until analysis.

An increase in HBD-1 production and/or activity after 5 days of approximately 4 pg/mL in the Upper Layers was observed for the hand sanitizer with Lipigenine™ as compared to the hand sanitizer without Lipigenine™. Additionally, a statistically significant (95% confidence) increase in HBD-1 production and/or activity after 10 days of about 35 pg/nL in the Lower Layers was observed for the hand sanitizer with Lipigenine™ as compared to untreated skin. An increase in HBD-2 production and/or activity after 10 days of about 21 pg/mL in the Lower Layers was observed for the hand sanitizer with 1% Lipigenine™ as compared to a hand sanitizer without Lipigenine™. Additionally, a statistically significant (90% confidence) increase in HBD-2 production and/or activity after 10 days of about 48 pg/mL was observed in the Lower Layers. Finally, a statistically significant (90% confidence) increase in HBD-3 production and/or activity after 10 days of about 48 pg/mL was observed in the Upper Layers. These results are shown below in Table 4.

TABLE 4

| | | | |
|---|---|---|---|
| | (Reference) | (Sanitizer | (Standard |
| | PURELL | Control) | Control) |
| | Advanced FF w/1% | PURELL Advanced | Untreated Skin |
| | Lipigenine ™ used as | Gel FF used as a | Control |
| Layer/Day | a leave-on (Code-S) | leave-on (Code-E) | (Code-U) |

HBD-1 Concentration (pg/mL)
2-4 Upper Layers

| | | | |
|---|---|---|---|
| 0 days | 0 | 0 | 0 |
| 5 days | 4.946 | 0.829 | 0.000 |
| 10 days | 11.862 | 15.423 u | 0.000 |

5-7 Lower Layers

| | | | |
|---|---|---|---|
| 0 days | 0 | 0 | 0 |
| 5 days | 3.385 | 6.834 | 0.000 |
| 10 days | 34.98 U | 18.924 | 0.000 |

HBD-2 Concentration (pg/mL)
2-4 Upper Layers

| | | | |
|---|---|---|---|
| 0 days | 0 | 0 | 0 |
| 5 days | 16.937 | 12.121 | 0.000 |
| 10 days | 39.856 | 74.995 U | 0.000 |

5-7 Lower Layers

| | | | |
|---|---|---|---|
| 0 days | 0 | 0 | 0 |
| 5 days | 8.109 | 6.978 | 0.000 |
| 10 days | 48.056 u | 27.586 | 0.000 |

HBD-3 Concentration (pg/mL)
2-4 Upper Layers

| | | | |
|---|---|---|---|
| 0 days | 0 | 0 | 0 |
| 5 days | 63.735 | 64.371 | 0.000 |
| 10 days | 172.088 u | 268.432 U | 0.000 |

5-7 Lower Layers

| | | | |
|---|---|---|---|
| 0 days | 0 | 0 | 0 |
| 5 days | −4.215 | 63.710 | 0.000 |
| 10 days | 141.267 | 140.694 | 0.000 |

### Example 10

The 3D skin model EpiDerm™ was used to evaluate Lipigenine™ AMP stimulation efficacy in sanitizer.

Two sanitizing compositions were prepared: 1) PURELL Advanced gel (Control) and 2) PURELL Advanced gel including 0.1% Lipigenine™

The sanitizing compositions were applied to the top surface of 3D skin tissue 2× a day and 10× a day for a period of five days. After 24 hours treatment, the culture mediums wee collected into storage plates, labeled, and stored at −70° C. The tissues were fed with fresh medium and the treatments were repeated. The medium samples were stored and the tissues were fed daily (every 24 hours). After 5 days of this treatment protocol, MTT assays were performed to evaluate the tissue status and measure the test articles' cytotoxicity. The ELISA test was used for determining expression of HBDs and IL-1α.

### HBD-1

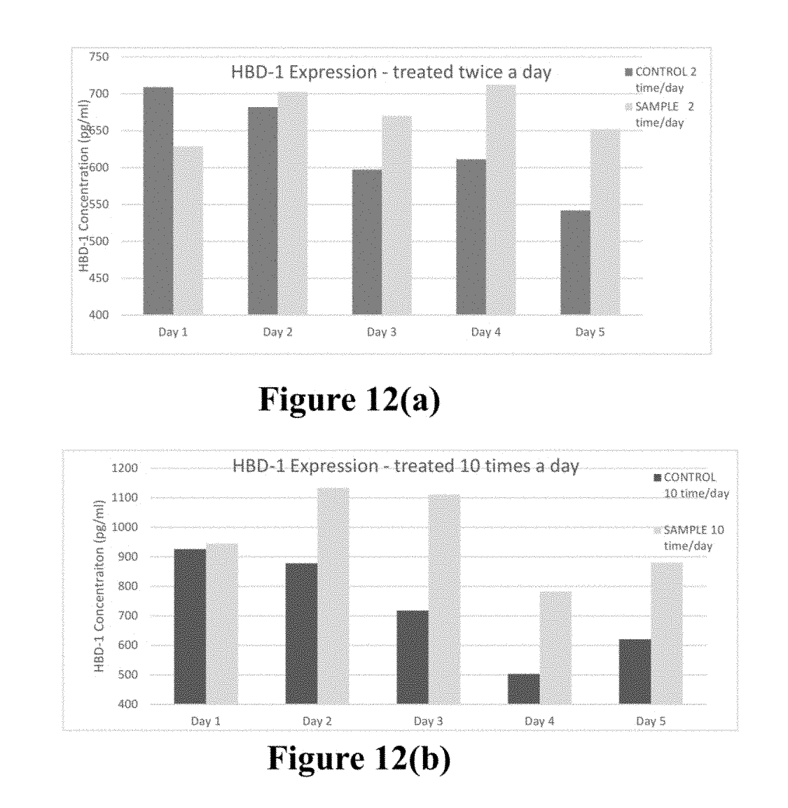

As illustrated in FIGS. 12(a) and 12(b), the addition of Lipigenine™ to the PURELL Advanced gel sanitizer (Sample) showed increased HBD-1 expression as compared to the Control after applying both 2× and 10× a day. In each case, the Sample showed an increase in HBD-1 expression over the Control starting at day 2, and each day thereafter (although with treatment 10× day the Sample showed an increase in HBD-1 expression day 1.

### HBD-2

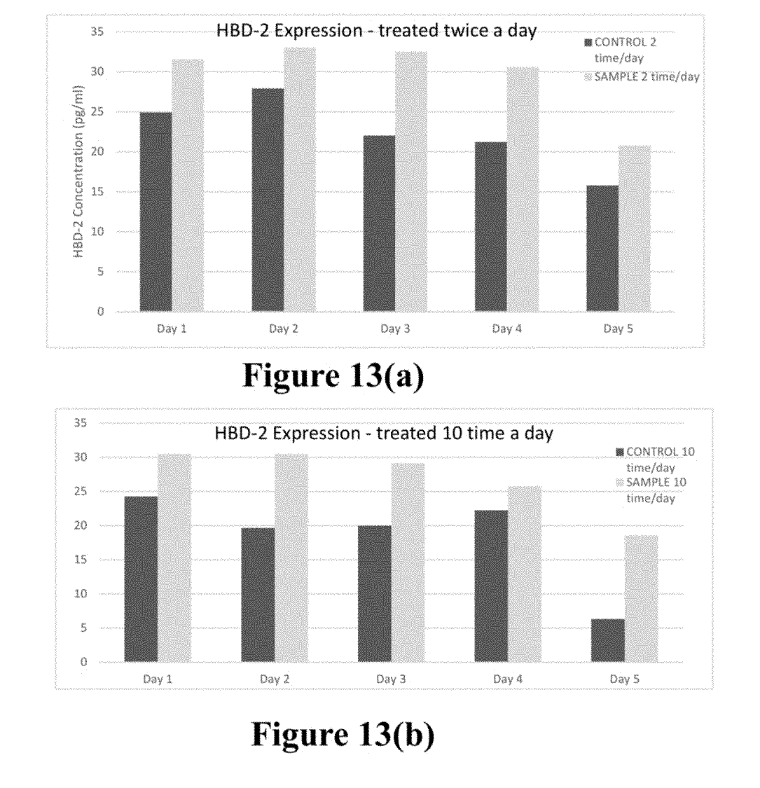

The results indicated that the alcohol in sanitizers may inhibit the expression of HBD-2 in tissue. However, the addition of Lipigenine™ to the sanitizer can bring the HBD-2 expression back to normal levels by treatment both 2× and 10× a day. As illustrated in FIGS. 13(a) and 13(b), the addition of Lipigenine™ to the PURELL Advanced gel sanitizer (Sample) showed increased HBD-2 expression as compared to the Control after applying both 2× and 10× a day. In each case, the Sample showed an increase in HBD-2 expression over the Control starting at day 1, and each day thereafter.

### HBD-3

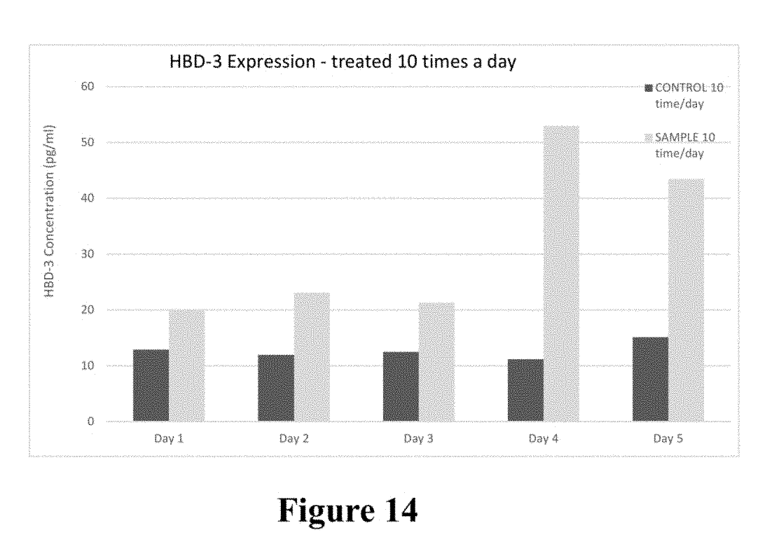

The results indicated that the alcohol in sanitizers may inhibit the expression of HBD-3 in tissue. However, the addition of Lipigenine™ to the sanitizer can bring the HBD-3 expression back to normal levels by treatment 10× a day after 4 days. As illustrated in FIG. 14, the addition of Lipigenine™ to the PURELL Advanced gel sanitizer (Sample) showed increased HBD-3 expression as compared to the Control after applying 10× a day. The Sample showed an increase in HBD-3 expression over the Control starting at day 1, and each day thereafter.

Although embodiments of the invention have been described herein, it should be appreciated that many modifications can be made without departing from the spirit and scope of the general inventive concepts. All such modifications are intended to be included within the scope of the invention, which is to be limited only by the following claims.

1. A sanitizing composition for stimulating the production and/or activity of antimicrobial peptides, the sanitizing composition comprising;
about 0.005 wt. % to about 15.0 wt. % of an active ingredient;
one or more ingredients that deliver a sanitizing effect;
wherein the active ingredient comprises one or more of an extract and a polypeptide, wherein the sanitizing composition increases the production and/or activity of at least one antimicrobial peptide by a statistically significant amount, as compared to an otherwise identical sanitizing composition without the active ingredient.

2. The sanitizing composition of claim 1, wherein the one or more ingredients that deliver a sanitizing effect is an anti-microbial ingredient.

3. The sanitizing composition of claim 2, wherein the anti-microbial ingredient is selected from one or more of alcohol and a quaternary ammonium compound.

4. (canceled)

5. (canceled)

6. The sanitizing composition of claim 1, wherein the one or more ingredients that deliver a sanitizing effect is present in an amount above about 70.0 wt. %, based on the weight of the total composition.

7. The sanitizing composition of claim 1, wherein the extract is one or more of a plant extract, a seed extract, and a fruit extract.

8. The sanitizing composition of claim 1, wherein the extract is a seed extract.

9. The sanitizing composition of claim 8, wherein the seed extract is at least one of linseed extract, flaxseed extract, hemp seed extract, grape seed extract, and grapefruit seed extract.

10. The sanitizing composition of claim 1, wherein the extract is a hydrolysate of proteins.

11. The sanitizing composition of claim 10, wherein the hydrolysate of proteins is a hydrolysate of proteins extracted from linseed seeds.

12. The sanitizing composition of claim 11, wherein the hydrolysate of linseed proteins contains from about 0.1 to about 5.0 g/l of peptide compounds and from about 0.1 to about 2.0 g/l of sugar.

13. The sanitizing composition of claim 12, wherein the peptide compounds have a molecular weight below about 5.0 kDa.

14. The sanitizing composition of claim 1, wherein the active ingredient is a polypeptide.

15. The sanitizing composition of claim 14, wherein the polypeptide is at least one of an oligopeptide and a hexapeptide.

16. The sanitizing composition of claim 1, wherein the sanitizing composition comprises from about 0.05 to about 5.0 wt. % of active ingredient, based on the weight of the sanitizing composition.

17. (canceled)

18. The sanitizing composition of claim 1, wherein the sanitizing composition further comprises one or more skin conditioning agents.

19. The sanitizing composition of claim 18, wherein the one or more skin conditioning agents comprises one or more humectants, comprising propylene glycol, hexylene glycol, 1,4-dihydroxyhexane, 1,2,6-hexanetriol, sorbitol, butylene glycol, caprylyl glycol, propanediols, such as methyl propane diol, dipropylene glycol, triethylene glycol, glycerin (glycerol), polyethylene glycols, ethoxydiglycol, polyethylene sorbitol, glyceryl caprylate/caprate, and combinations thereof.

20. (canceled)

21. (canceled)

22. (canceled)

23. (canceled)

24. The sanitizing composition of claim 1, wherein said sanitizing composition increases the production and/or activity of defensins by at least about 7%, relative to an otherwise identical sanitizing composition without said active ingredient.

25. The sanitizing composition of claim 1, wherein the sanitizing composition increases the production and/or activity of defensins by at least about 18%, relative to an otherwise identical sanitizing composition without the active ingredient.

26. The sanitizing composition of claim 1, wherein the sanitizing composition increases the production and/or activity of defensins by at least about 20%, relative to an otherwise identical sanitizing composition without the active ingredient.

27. The sanitizing composition of claim 1, wherein the sanitizing composition increases the production and/or activity of cathelicidin-related antimicrobial peptides by at least about 32%, relative to an otherwise identical sanitizing composition without the active ingredient.

28. The sanitizing composition of claim 1, wherein the sanitizing composition decreases the production and/or activity of pro-inflammatory markers by at least about 30%, relative to an otherwise identical sanitizing composition without the active ingredient.

29. The sanitizing composition of claim 1, wherein the sanitizing composition increases the production and/or activity of defensins by at least about 1 pg/mL, relative to an otherwise identical sanitizing composition without the active ingredient.

30. The sanitizing composition of claim 1, wherein the sanitizing composition increases the production and/or activity of defensins by at least about 4 pg/mL, relative to an otherwise identical sanitizing composition without the active ingredient.

31. (canceled)

32. (canceled)

33. A method of skin treatment to increase the production and/or activity of at least one antimicrobial peptide on the skin, the method comprising:
applying a sanitizing composition to a skin surface, wherein the sanitizing composition comprises:
about 0.005 wt. % to about 15.0 wt. % of an active ingredient;
one or more ingredients that deliver a sanitizing effect;
wherein the active ingredient comprises one or more of an extract and a polypeptide, where the sanitizing composition increases the production and/or activity of at least one antimicrobial peptide by a statistically significant amount, as compared to an otherwise identical sanitizing composition without the active ingredient.

34. (canceled)

35. (canceled)

36. A sanitizing composition for increasing the innate immunity of the skin comprising;
about 0.005 wt. % to about 15.0 wt. % of an active ingredient comprising one or more of an extract and a polypeptide;
about 40.0 wt. % to about 95.0 wt. % of one or more ingredients that deliver a sanitizing effect;
about 0.01 wt. % to about 5.0 wt. % of a foaming agent; and
about 0.01 wt. % to about 10.0 wt. % of one or more skin-conditioners.

37. A nasal spray composition, comprising:
about 0.005 wt. % to about 15.0 wt. % of an active ingredient; and
one or more C₁₋₈ alcohols
wherein the active ingredient comprises one or more of a probiotic, probiotic derivative, and a prebiotic and wherein the sanitizing composition reduces pathogen binding in the nose by a statistically significant amount, as compared to an otherwise identical nasal spray without the active ingredient.

38. (canceled)
